(19) United States
(12) Patent Application Publication
(10) Pub. No.: US 2010/0105629 A1
(43) Pub. Date: Apr. 29, 2010

(54) N-Substituted Peptidomimetic Inhibitors of Dipeptidylpeptidase IV

(76) Inventors: William W. Bachovchin, Cambridge, MA (US); Hung-Sen Lai, Andover, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/531,755

(22) PCT Filed: Mar. 24, 2008
(86) PCT No.: PCT/US08/58003

§ 371 Date: Dec. 29, 2009

Related U.S. Application Data
(60) Provisional application No. 60/896,568, filed on Mar. 23, 2007.

(51) Int. Cl. A61K 38/05; C07F 5/02; A61K 31/69; C12N 9/99

(52) U.S. Cl. 514/19; 562/7; 514/64; 435/184

(57) ABSTRACT

One aspect of the present invention relates to inhibitors of post-pro line protease enzymes, such as inhibitors of dipeptidyl peptidase IV (DPIV). Other aspects of the invention relate to pharmaceutical compositions and methods of use thereof. In certain embodiments, a DPIV inhibitor comprises a carboxylic acid-containing side chain moiety at the P1 or P2 position or both. The inhibitors of the invention should possess improved specificity for the targeted protease and low toxicity.

### RELATED APPLICATIONS

This application claims the benefit of priority to United States Provisional Patent Application Ser. No. 60/896,568, filed Mar. 23, 2007.

### BACKGROUND OF THE INVENTION

Proteases are enzymes that cleave proteins at specific peptide bonds. Proteases can be classified into four generic classes: serine; thiol or cysteinyl; acid or aspartyl; and metalloproteases (Cuypers et al., J. Biol. Chem. 1982, 257, 7086). Proteases are essential to a variety of biological activities, such as digestion, formation and dissolution of blood clots, reproduction, and immune reaction to foreign cells and organisms. However, aberrant proteolysis is associated with a number of diseases in humans and other mammals. Accordingly, it is often beneficial to disrupt the function of one or more proteolytic enzymes in the course of treating a patient.

The binding site for a peptide substrate consists of a series of “specificity subsites” across the surface of the enzyme. The term “specificity subsite” refers to a pocket or other site on the enzyme capable of interacting with a portion of a substrate for the enzyme. In discussing the interactions of peptides with proteases, e.g., serine and cysteine proteases, the present application utilizes the nomenclature of Schechter and Berger (Biochem. Biophys. Res. Commun. 1967, 27, 157-162). The individual amino acid residues of a substrate or inhibitor are designated P1, P2, etc. and the corresponding subsites of the enzyme are designated S1, S2, etc., starting with the carboxy terminal residue produced in the cleavage reaction. The scissile bond of the substrate is the amide bond between P1-P1′ of the substrate. Thus, for a peptide Xaa1-Xaa2-Xaa3-Xaa4, which is cleaved between the Xaa3 and Xaa4 residues, the Xaa3 residue is referred to as the P1 residue and binds to the S1 subsite of the enzyme, Xaa2 is referred to as the P2 residue and binds to the S2 subsite, and so forth.

Dipeptidyl peptidase IV (DPIV or DPPIV) is a serine protease that cleaves N-terminal dipeptides from a peptide chain containing, preferably, a proline residue in the penultimate position, i.e., in the P1 position. DPIV belongs to a group of cell-membrane-associated peptidases and, like the majority of cell-surface peptidases, is a type II integral membrane protein, being anchored to the plasma membrane by its signal sequence. DPIV is found in a variety of differentiated mammalian epithelia, endothelia and hematopoetic cells and tissues, including those of lymphoid origin where it is found specifically on the surface of CD4⁺ T cells. DPIV has been identified as leukocyte differentiation marker CD26.

### SUMMARY OF THE INVENTION

One aspect of the present invention relates to inhibitors of post-proline protease enzymes, such as inhibitors of dipeptidyl peptidase IV, as well as pharmaceutical compositions thereof, and methods for using such inhibitors. In certain embodiments, the inhibitors of the present invention comprise particular classes of sidechains containing a carboxylic acid moiety in the P1 and/or P2 position of the inhibitor. In certain embodiments, the compounds of the present invention should have a desirable therapeutic index, due in part to reduced toxicity and/or improved specificity for the targeted protease.

One aspect of the invention relates to a protease inhibitor represented by:

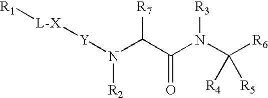

or a pharmaceutically acceptable salt thereof;

wherein
R₁ is selected from the group consisting of H, alkyl, alkoxy, alkenyl, alkynyl, amino, alkylamino, acylamino, cyano, sulfonylamino, acyloxy, aryl, cycloalkyl, heterocyclyl, heteroaryl, and polypeptide chains of 1 to 8 amino acid residues;
R₂ is selected from the group consisting of H, lower alkyl, and aralkyl;
R₃ is selected from the group consisting of

lower alkyl;

R₃₁R₃₂N(CH₂)ₘ—, wherein R₃₁ is a pyridinyl or pyrimidinyl moiety optionally mono- or independently disubstituted with (C₁₋₄alkyl, (C₁₋₄alkoxy, halogen, trifluoromethyl, cyano, or nitro; or phenyl optionally mono- or disubstituted with (C₁₋₄)alkyl, (C₁₋₄)alkoxy or halogen; and R₃₂ is selected from hydrogen and (C₁₋₈)alkyl; and m is 2 or 3;

(C₃₋₁₂)cycloalkyl optionally monosubstituted in the 1-position with

(C₁₋₃)hydroxyalkyl;

R₃₃(CH₂)ₙ—, wherein either R₃₃ is selected from the group consisting of phenyl optionally mono- or independently di- or independently trisubstituted with (C₁₋₄)alkyl, (C₁₋₄)alkoxy, halogen, or phenylthio optionally monosubstituted in the phenyl ring with hydroxymethyl or (C₁₋₈)alkyl; [3.1.1]-bicyclic carbocyclic moiety optionally substituted with (C₁₋₈)alkyl; pyridinyl or naphthyl moiety optionally mono- or disubstituted with (C₁₋₄)alkyl, (C₁₋₄)alkoxy or halogen; cyclohexene; and adamantyl; and n is 1 to 3; or R₃₃ is phenoxy optionally mono- or disubstituted with (C₁₋₄)alkyl, (C₁₋₄)alkoxy or halogen; and n is 2 or 3;

(R₃₄)₂CH(CH₂)₂—, wherein R₃₄ independently is phenyl optionally mono- or disubstituted with (C₁₋₄)alkyl, (C₁₋₄)alkoxy or halogen;

(R₃₅)₂(CH₂)ₚ—, wherein R₃₅ is 2-oxopyrrolidinyl or (C₂₋₄)alkoxy and p is 2 to 4; and

R₃₆ is selected from indanyl; a pyrrolidinyl or piperidinyl moiety optionally substituted with benzyl; a [2.2.1]- or [3.1.1]bicyclic carbocyclic moiety optionally substituted with (C₁₋₈)alkyl; adamantyl; and (C₁₋₈)alkyl optionally substituted with hydroxy, hydroxymethyl, or phenyl optionally substituted with (C₁₋₄)alkyl, (C₁₋₄)alkoxy, or halogen;

R₄ is selected from the group consisting of H, halogen, and lower alkyl;
R₅ is selected from the group consisting of H, halogen, lower alkyl, and aralkyl;
R₆ is selected from the group consisting of —CN, —CHO, —CH═NR₆₃,

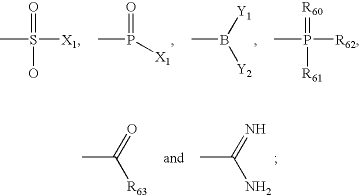

wherein

R₆₃ represents H, alkyl, alkenyl, alkynyl, —C(X₁)(X₂)(X₃), —(CH₂)ₘ—R₆₄, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R₆₄, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R₆₄, —C(O)C(O)NH₂, or —C(O)C(O)OR₆₅;

R₆₄ represents independently for each occurrence a substituted or unsubstituted aryl, aralkyl, cycloalkyl, cycloalkenyl, or heterocycle;

R₆₅ represents independently for each occurrence hydrogen, or a substituted or unsubstituted alkyl, alkenyl, aryl, aralkyl, cycloalkyl, cycloalkenyl, or heterocycle;

Y₁ and Y₂ independently represent OH, or a group capable of being hydrolyzed to a hydroxyl group; or taken together with the boron to which they are bonded form a 5-membered to 8-membered ring comprising said boron and two oxygen atoms bonded to said boron

R₆₀ is O or S;

R₆₁ is N₃, SH, NH₂, NO₂ or —OR₇;

R₆₂ is selected from the group consisting of hydrogen, lower alkyl, amine, and —OR₆₅, or a pharmaceutically acceptable salt; or R₆₁ and R₆₂ taken together with the phosphorous atom to which they are attached complete a heterocyclic ring having from 5 to 8 atoms in the ring structure;

X₁ is halogen;

X₂ and X₃ is hydrogen or halogen; and

m is zero or an integer in the range of 1 to 8; and

n is an integer in the range of 1 to 8;

R₇ is selected from the group consisting of alkyl, alkoxy, alkenyl, alkynyl, aminoalkyl, aminoacyl, acyloxy, aryl, aralkyl, cycloalkyl, heterocyclyl, heteroaryl, and heteroaralkyl;
R₈ is selected from the group consisting of H, aryl, alkyl, aralkyl, cycloalkyl, heterocyclyl, heteroaryl, heteroaralkyl, and polypeptide chains of 1 to 8 amino acid residues;
L is absent or selected from the group consisting of alkyl, alkenyl, alkynyl, (CH₂)ₘO(CH₂)ₘ—, —(CH₂)ₘNR₂(CH₂)ₘ—, and —(CH₂)ₘS(CH₂)ₘ—, wherein m is, independently for each occurrence, an integer from 0 to 10; and n is an integer from 1 to 6;
X is absent or selected from the group consisting of —N(R₈)—, —O—, and —S—; and
Y is absent or is selected from —C(═O)—, —C(═S)—, and —SO₂—.

Another aspect of the invention provides a pharmaceutical composition, comprising a pharmaceutically acceptable carrier; and a subject protease inhibitor or a pharmaceutically acceptable salt thereof.

Another aspect of the invention provides for use of one or more of the subject inhibitors in the manufacture of a medicament for inhibiting a post-proline protease enzyme in vivo. For example, the subject inhibitors can be used to manufacture medicaments for increasing plasma concentrations of one or more peptide hormones processed by post-proline protease enzymes (e.g., DPIV and the like). Exemplary medicaments are useful in increasing plasma concentrations of such hormones as glucagons-like peptide, NPY, PPY, secretin, GLP-1, GLP-2, and GIP.

Yet another aspect of the invention provides a packaged pharmaceutical comprising: a preparation of one or more of the subject protease inhibitors; a pharmaceutically acceptable carrier; and instructions, written and/or pictorial, describing the use of the preparation for inhibiting a post-proline protease enzyme in vivo, such as for regulating glucose metabolism.

### DETAILED DESCRIPTION OF THE INVENTION

The present invention relates to inhibitors of post-proline proteases, such as inhibitors of dipeptidyl peptidase IV, as well as pharmaceutical compositions thereof, and methods for using such inhibitors. In certain embodiments, the inhibitors have an acidic amino acid and an electrophilic site comprising one of a variety of side chains.

Salient features for compounds of the present invention include: better therapeutic indices, owing in part to reduced toxicity and/or improved specificity for the targeted protease; better oral availability; increased shelf-life; and/or increased duration of action (such as single oral dosage formulations which are effective for more than 4 hours, and even more preferably for more than 8, 12, or 16 hours).

The compounds of the present invention can be used as part of treatments for a variety of disorders/conditions, such as those which are mediated by DPIV. For instance, the subject inhibitors can be used to up-regulate GIP and GLP-1 activities, e.g., by increasing the half-life of those hormones; as part of a treatment for regulating glucose levels and/or metabolism, e.g., to reduce insulin resistance; to treat hyperglycemia, hyperinsulinemia, obesity, hyperlipidemia, hyperlipoproteinemia (such as chylomicrons, VLDL and LDL); and to regulate body fat and more generally lipid stores; and, more generally, for the improvement of metabolism disorders, especially those associated with diabetes, obesity and/or atherosclerosis.

One aspect of the invention relates to protease inhibitors represented by:

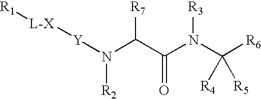

or a pharmaceutically acceptable salt thereof;

wherein
R₁ is selected from the group consisting of H, alkyl, alkoxy, alkenyl, alkynyl, amino, alkylamino, acylamino, cyano, sulfonylamino, acyloxy, aryl, cycloalkyl, heterocyclyl, heteroaryl, and polypeptide chains of 1 to 8 amino acid residues;
R₂ is selected from the group consisting of H, lower alkyl, and aralkyl;
R₃ is selected from the group consisting of

lower alkyl;

R₃₁R₃₂N(CH₂)ₘ—, wherein R₃₁ is a pyridinyl or pyrimidinyl moiety optionally mono- or independently disubstituted with (C₁₋₄)alkyl, (C₁₋₄)alkoxy, halogen, trifluoromethyl, cyano, or nitro; or phenyl optionally mono- or disubstituted with (C₁₋₄)alkyl, (C₁₋₄)alkoxy or halogen; and R₃₂ is selected from hydrogen and (C₁₋₈)alkyl; and m is 2 or 3;

(C₃₋₁₂)cycloalkyl optionally monosubstituted in the 1-position with

(C₁₋₃)hydroxyalkyl;

R₃₃(CH₂)ₙ—, wherein either R₃₃ is selected from the group consisting of phenyl optionally mono- or independently di- or independently trisubstituted with (C₁₋₄)alkyl, (C₁₋₄)alkoxy, halogen, or phenylthio optionally monosubstituted in the phenyl ring with hydroxymethyl or (C₁₋₈)alkyl; [3.1.1]-bicyclic carbocyclic moiety optionally substituted with (C₁₋₈)alkyl; pyridinyl or naphthyl moiety optionally mono- or disubstituted with (C₁₋₄)alkyl, (C₁₋₄)alkoxy or halogen; cyclohexene; and adamantyl; and n is 1 to 3; or R₃₃ is phenoxy optionally mono- or disubstituted with (C₁₋₄)alkyl, (C₁₋₄)alkoxy or halogen; and n is 2 or 3;

(R₃₄)₂CH(CH₂)₂—, wherein R₃₄ independently is phenyl optionally mono- or disubstituted with (C₁₋₄)alkyl, (C₁₋₄)alkoxy or halogen;

(R₃₅)₂(CH₂)ₚ—, wherein R₃₅ is 2-oxopyrrolidinyl or (C₂₋₄)alkoxy and p is 2 to 4; and

R₃₆ is selected from indanyl; a pyrrolidinyl or piperidinyl moiety optionally substituted with benzyl; a [2.2.1]- or [3.1.1]bicyclic carbocyclic moiety optionally substituted with (C₁₋₈)alkyl; adamantyl; and (C₁₋₈)alkyl optionally substituted with hydroxy, hydroxymethyl, or phenyl optionally substituted with (C₁₋₄)alkyl, (C₁₋₄)alkoxy, or halogen;

R₄ is selected from the group consisting of H, halogen, and lower alkyl;
R₅ is selected from the group consisting of H, halogen, lower alkyl, and aralkyl;
R₆ is selected from the group consisting of —CN, —CHO, —CH═NR₆₃,

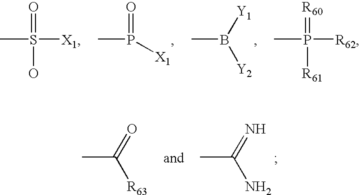

wherein

R₆₃ represents H, alkyl, alkenyl, alkynyl, —C(X₁)(X₂)(X₃), —(CH₂)ₘ—R₆₄, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R₆₄, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R₆₄, —C(O)C(O)NH₂, or —C(O)C(O)OR₆₅;

R₆₄ represents independently for each occurrence a substituted or unsubstituted aryl, aralkyl, cycloalkyl, cycloalkenyl, or heterocycle;

R₆₅ represents independently for each occurrence hydrogen, or a substituted or unsubstituted alkyl, alkenyl, aryl, aralkyl, cycloalkyl, cycloalkenyl, or heterocycle;

Y₁ and Y₂ independently represent OH, or a group capable of being hydrolyzed to a hydroxyl group; or taken together with the boron to which they are bonded form a 5-membered to 8-membered ring comprising said boron and two oxygen atoms bonded to said boron;

R₆₀ is O or S;

R₆₁ is N₃, SH, NH₂, NO₂ or —OR₇;

R₆₂ is selected from the group consisting of hydrogen, lower alkyl, amine, and —OR₆₅, or a pharmaceutically acceptable salt; or R₆₁ and R₆₂ taken together with the phosphorous atom to which they are attached complete a heterocyclic ring having from 5 to 8 atoms in the ring structure;

X₁ is halogen;

X₂ and X₃ is hydrogen or halogen; and

m is zero or an integer in the range of 1 to 8; and

n is an integer in the range of 1 to 8;

R₇ is selected from the group consisting of alkyl, alkoxy, alkenyl, alkynyl, aminoalkyl, aminoacyl, acyloxy, aryl, aralkyl, cycloalkyl, heterocyclyl, heteroaryl, and heteroaralkyl;
R₈ is selected from the group consisting of H, aryl, alkyl, aralkyl, cycloalkyl, heterocyclyl, heteroaryl, heteroaralkyl, and polypeptide chains of 1 to 8 amino acid residues;
L is absent or selected from the group consisting of alkyl, alkenyl, alkynyl, (CH₂)ₘO(CH₂)ₘ—, —(CH₂)ₘNR₂(CH₂)ₘ—, and —(CH₂)ₘS(CH₂)ₘ—, wherein m is, independently for each occurrence, an integer from 0 to 10; and n is an integer from 1 to 6;
X is absent or selected from the group consisting of —N(R₈)—, —O—, and —S—; and
Y is absent or is selected from —C(═O)—, —C(═S)—, and —SO₂—.

In certain embodiments, R₆ is CN, CHO, or

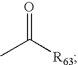

### and R₆₃ is C(X₁)(X₂)(X₃).

In other embodiments, R₆ is

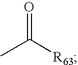

R₆₃ is C(X₁)(X₂)(X₃); X₁ is fluorine; and X₂ and X₃ are independently selected from the group consisting of H and fluorine.

In other embodiments, R₆ is a group of formula —B(Y₁)(Y₂), wherein Y₁ and Y₂ independently represent OH or a group capable of being hydrolyzed to a hydroxyl group; or taken together with the boron to which they are bonded form a 5-membered to 8-membered ring comprising said boron and two oxygen atoms bonded to said boron.

In certain embodiments, the inhibitor is represented by the formula:

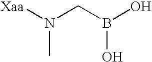

or a pharmaceutically acceptable salt thereof;

wherein Xaa is selected from the group consisting of Trp, Pro, Glu, Gly, Val, Aad, Arg, 1-Naphthyl-Ala, Chg (cyclohexylglycine), Ile, t-Leu, Ethly-Gly, Phe, Lys, Met, Tyr, Ala, n-Propyl-Gly, Thr, Leu, Gln, Ser, Asn, Asp, His, Methyl-Gly, Ethyl-Gly, t-Butyl-Gly, Methyl-Ala, Aib, N-Methyl-Gly, N-Ethyl-Gly, N-t-Butyl-Gly, and N-Methyl-Ala.

In other embodiments, the inhibitor is represented by the formula:

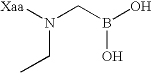

or a pharmaceutically acceptable salt thereof;

wherein Xaa is selected from the group consisting of Trp, Pro, Glu, Gly, Val, Aad, Arg, 1-Naphthyl-Ala, Chg (Cyclohexylglycine), Ile, t-Leu, Ethyl-Gly, Phe, Lys, Tyr, Ala, n-Propyl-Gly, Leu, Gln, Ser, Asn, Asp, His, Aib, N-Methyl-Gly, and N-Ethyl-Gly.

In certain embodiments, the inhibitor has a therapeutic index in humans (e.g., such as a therapeutic index for regulating glucose metabolism) of at least 2, at least 5, at least 10, or at least 100.

Another aspect of the invention provides a pharmaceutical composition, comprising a pharmaceutically acceptable carrier; and a subject protease inhibitor or a pharmaceutically acceptable salt thereof.

Another aspect of the invention provides for use of one or more of the subject inhibitors in the manufacture of a medicament for inhibiting a post-proline protease enzyme in vivo. For example, the subject inhibitors can be used to manufacture medicaments for increasing plasma concentrations of one or more peptide hormones processed by post-proline protease enzymes (e.g., DP-IV and the like). Exemplary medicaments are useful in increasing plasma concentrations of hormones such as glucagon-like peptide, NPY, PPY, secretin, GLP-1, GLP-2, and GIP.

In certain embodiments, the subject inhibitors may be used to manufacture a medicament for regulating glucose metabolism, such as for treating patients suffering from Type II diabetes, insulin resistance, glucose intolerance, hyperglycemia, hypoglycemia, hyperinsulinemia, obesity, hyperlipidemia, or hyperlipoproteinemia.

Yet another aspect of the invention provides a packaged pharmaceutical product, comprising a preparation of one or more of the subject protease inhibitors; a pharmaceutically acceptable carrier; and written and/or pictorial instructions regarding the use of the preparation for inhibiting a post-proline protease enzyme in vivo, such as for regulating glucose metabolism.

The packaged pharmaceutical can also include, e.g., as a co-formulation with the protease inhibitor, or simply co-packaged therewith, insulin and/or an insulinotropic agent.

The packaged pharmaceutical can also include, e.g., as a co-formulation with the protease inhibitor, or simply co-packaged therewith, an M1 receptor antagonist, a prolactin inhibitor, agents acting on the ATP-dependent potassium channel of β-cells, metformin, and/or glucosidase inhibitors.

The present invention also relates to improved methods for the long-term reduction and abatement of at least one of the foregoing disorders based on a therapeutic regimen administered over the short-term.

The present invention further provides a method for regulating and altering on a long-term basis the glucose and lipogenic responses of vertebrate animals, including humans.

In particular, the compounds of the invention may be employed to provide methods for producing long-lasting beneficial changes in one or more of the following: the sensitivity of the cellular response of a species to insulin (reduction of insulin resistance), blood insulin levels, hyperinsulinemia, blood glucose levels, the amount of body fat stores, and blood lipoprotein levels, thus providing effective treatments for diabetes, obesity and/or atherosclerosis.

While not wishing to be bound by any particular theory, it is observed that compounds which inhibit DPIV are, correlatively, able to improve glucose tolerance, though not necessarily through mechanisms involving DPIV inhibition per se. Indeed, similar compounds have been shown to be effective in mice lacking a GLP-1 receptor suggesting that the subject method may include a mechanism of action not directly implicating GLP-1, though it has not been ruled out that GLP-1 may have other receptors. However, in light of the correlation with DPIV inhibition, in certain embodiments, the subject method utilizes an agent with a Kᵢ for DPIV inhibition of 50.0 nM or less; of 10.0 nM or less; or of 1.0, 0.1, or 0.01 nM or less. Indeed, inhibitors with Kᵢ values in the picomolar and femtomolar range are contemplated. Thus, while the active agents are described herein, for convenience, as “DPIV inhibitors”, it will be understood that such nomenclature is not intending to limit the subject invention to a particular mechanism of action.

Certain of the subject compounds have extended duration of therapeutic action. Accordingly, in certain embodiments, the inhibitor(s) is selected, and the amount of inhibitor formulated, to provide a dosage which inhibits a serum post-proline protease (e.g., DPIV) by at least 50 percent for at least 4 hours after a single dose, for at least 8 hours after a single dose, or for 12 or 16 hours after a single dose.

For instance, in certain embodiments the method involves administration of a DPIV inhibitor, preferably at a predetermined time(s) during a 24-hour period, in an amount effective to improve one or more aberrant indices associated with glucose metabolism disorders (e.g., glucose intolerance, insulin resistance, hyperglycemia, hyperinsulinemia, and Type I and II diabetes).

In other embodiments, the method involves administration of a DPIV inhibitor in an amount effective to improve aberrant indices associated with obesity. Fat cells release the hormone leptin, which travels in the bloodstream to the brain and, through leptin receptors there, stimulates production of GLP-1. GLP-1, in turn, produces the sensation of being full. The leading theory is that the fat cells of most obese people probably produce enough leptin, but leptin may not be able to engage properly the leptin receptors in the brain, and so does not stimulate production of GLP-1. Therefore, there is a great deal of research towards utilizing preparations of GLP-1 as an appetite suppressant. The subject method provides a means for increasing the half-life of both endogenous and ectopically added GLP-1 in the treatment of disorders associated with obesity.

In a more general sense, the present invention provides methods and compositions for altering the pharmacokinetics of a variety of different polypeptide hormones by inhibiting the proteolysis of one or more peptide hormones by DPIV or another proteolytic activity. Post-secretory metabolism is an important element in the overall homeostasis of regulatory peptides, and the other enzymes involved in these processes may be suitable targets for pharmacological intervention by the subject method.

In certain instances, the subject method can be used to increase the half-life of other proglucagon-derived peptides, such as glicentin (corresponding to PG 1-69), oxyntomodulin (PG 33-69), glicentin-related pancreatic polypeptide (GRPP, PG 1-30), intervening peptide-2 (IP-2, PG 111-122 amide), and glucagon-like peptide-2 (GLP-2, PG 126-158). GLP-2, for example, has been identified as a factor responsible for inducing proliferation of intestinal epithelium. See, e.g., Drucker et al. Proc. Natl. Acad. Sci. USA 1996, 93, 7911. The DPP IV inhibitors can also be used as part of a regimen for treating injury, inflammation or resection of intestinal tissue, e.g., where enhanced growth and repair of the intestinal mucosal epithelial is desired, such as in the treatment of Crohn's disease or Inflammatory Bowel Disease (IBD).

Another aspect of the invention relates to a method of treating growth hormone deficient children or improving nutrition or altering body composition (muscle vs. fat) in adults. DPP IV has been implicated in the metabolism and inactivation of growth hormone-releasing factor (GHRF). GHRF is a member of a family of homologous peptides that includes glucagon, secretin, vasoactive intestinal peptide (VIP), peptide histidine isoleucine (PHI), pituitary adenylate cyclase activating peptide (PACAP), gastric inhibitory peptide (GIP) and helodermin. Kubiak et al. Peptide Res. 1994, 7, 153. GHRF is secreted by the hypothalamus, and stimulates the release of growth hormone (GH) from the anterior pituitary. The subject method can also be used in veterinary practice, for example, to develop higher yield milk production, and higher yield, leaner livestock.

The DPP IV inhibitors of the invention can be used to alter the plasma half-life of secretin, VIP, PHI, PACAP, GIP and/or helodermin. In certain instances, the inhibitors can also be used to alter the pharmacokinetics of Peptide YY and neuropeptide Y, both members of the pancreatic polypeptide family, because DPP IV has been implicated in the processing of those peptides in a manner which alters receptor selectivity. In other embodiments, the DPP IV inhibitors can be used to stimulate hematopoiesis. In still other embodiments, the DPP IV inhibitors can be used to inhibit growth or vascularization of transformed cells/tissues, e.g., to inhibit cell proliferation, such as that associated with tumor growth and metastasis, and for inhibiting angiogenesis in an abnormal proliferative cell mass. In other embodiments, the subject DPP IV inhibitors can be used to reduce immunological responses, i.e., as an immunosuppressant.

In still other examples, the DPP IV inhibitors according to the present invention can be used to treat CNS maladies, such as strokes, tumors, ischemia, Parkinson's disease, memory loss, hearing loss, vision loss, migraines, brain injury, spinal cord injury, Alzheimer's disease and amyotrophic lateral sclerosis (which has a CNS component). Additionally, the DPP IV inhibitors can be used to treat disorders having a more peripheral nature, including multiple sclerosis and diabetic neuropathy.

Another aspect of the present invention provides a method for stimulating hematopoietic cells in culture or in vivo. In certain embodiments, the subject DPP IV pro-inhibitors include an address moiety that is a substrate for a protease that is expressed in bone marrow. The DPP IV inhibitors of the invention can be used to address or to prevent a deficiency in hematopoietic cell number in a subject. Such deficiencies can arise, for example, from genetic abnormalities, disease, stress, chemotherapy, and radiation treatment.

In certain embodiments, the present invention provides a method of reducing the rate of degradation of tumor suppressors. In other embodiments, compounds of the present invention inhibit the growth of cancer cells. In other embodiments, the compounds of the present invention can be formulated in topical form for treatment of skin disorders. Such inhibitors are contemplated as possessing important practical application in treating cell proliferative diseases, such as cancer, restenosis, and psoriasis.

In other embodiments, the subject inhibitors can be used to inhibit growth or vascularization of transformed cells/tissues, e.g., to inhibit cell proliferation, such as that associated with tumor growth and metastasis, and for inhibiting angiogenesis in an abnormal proliferative cell mass.

In still other embodiments, the subject inhibitors can be used to reduce immunological responses, i.e., as an immunosuppressant.

Another aspect of the present invention relates to pharmaceutical compositions of the subject post-proline protease enzyme inhibitors, particularly DPIV inhibitors, and their uses in treating and/or preventing disorders which can be improved by altering the homeostasis of peptide hormones. In a certain embodiment, the inhibitors have hypoglycemic and antidiabetic activities, and can be used in the treatment of disorders marked by aberrant glucose metabolism (including storage). In particular embodiments, the compositions of the subject methods are useful as insulinotropic agents, or to potentiate the insulinotropic effects of such molecules as GLP-1. In this regard, certain embodiments of the present compositions can be useful for the treatment and/or prophylaxis of a variety of disorders, including one or more of: hyperlipidemia, hyperglycemia, obesity, glucose tolerance insufficiency, insulin resistance, and diabetic complications.

In general, the inhibitors of the subject method are small molecules, e.g., with molecular weights less than 7,500 amu, less than 5,000 amu, less than 2,000 amu, or less than 1,000 amu. In certain embodiments, the inhibitors are orally active.

### (i) Definitions

The term “boro-Ala” refers to the analog of alanine in which the carboxylate group (COOH) is replaced with a boronyl group (B(OH)₂). Likewise, the term “boro-Pro” refers to the analog of proline in which the carboxylate group (COOH) is replaced with a boronyl group (B(OH)₂). More generally, the term “boro-Xaa”, where Xaa is an amino acid residue, refers to the analog of an amino acid in which the carboxylate group (COOH) is replaced with a boronyl group (B(OH)₂).

A “patient” or “subject” to be treated by the subject method can mean either a human or non-human subject.

The term “ED₅₀” means the dose of a drug that, in 50% of patients, will provide a clinically relevant improvement or change in a physiological measurement, such as glucose responsiveness, increase in hematocrit, decrease in tumor volume, etc.

The term “IC₅₀” means the dose of a drug that inhibits a biological activity by 50%, e.g., the amount of inhibitor required to inhibit at least 50% of DPIV (or other PPCE) activity in vivo.

As used herein, the term “inhibitor” is meant to describe a compound that blocks or reduces an activity of an enzyme (for example, inhibition of proteolytic cleavage of standard fluorogenic peptide substrates such as suc-LLVY-AMC, Box-LLR-AMC and Z-LLE-AMC, inhibition of various catalytic activities of the 20S proteasome). An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme.

A compound is said to have an “insulinotropic activity” if it is able to stimulate, or cause the stimulation of, the synthesis or expression of the hormone insulin.

The term “interact” as used herein is meant to include all interactions (e.g., biochemical, chemical, or biophysical interactions) between molecules, such as protein-protein, protein-nucleic acid, nucleic acid-nucleic acid, protein-small molecule, nucleic acid-small molecule, or small molecule-small molecule interactions.

The term “LD₅₀” means the dose of a drug that is lethal in 50% of test subjects.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “therapeutic index” refers to the therapeutic index of a drug defined as LD₅₀/ED₅₀.

A “therapeutically effective amount” of a compound, e.g., such as a DPIV inhibitor of the present invention, with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

A “single oral dosage formulation” is a dosage which provides an amount of drug to produce a serum concentration at least as great as the EC₅₀ for that drug, but less than the LD₅₀. Another measure for a single oral dosage formulation is that it provides an amount of drug necessary to produce a serum concentration at least as great as the IC₅₀ for that drug, but less than the LD₅₀. By either measure, a single oral dosage formulation is preferably an amount of drug which produces a serum concentration at least 10 percent less than the LD₅₀, and even more preferably at least 50 percent, 75 percent, or even 90 percent less than the drug's the LD₅₀.

As used herein, the term “inhibitor” is meant to describe a compound that blocks or reduces an activity of an enzyme (for example, inhibition of proteolytic cleavage of standard fluorogenic peptide substrates such as suc-LLVY-AMC, Box-LLR-AMC and Z-LLE-AMC, inhibition of various catalytic activities of the 20S proteasome). An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme.

The term “C_{x-y}alkyl” refers to substituted or unsubstituted saturated hydrocarbon groups, including straight-chain alkyl and branched-chain alkyl groups that contain from x to y carbons in the chain, including haloalkyl groups, such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

Unless the number of carbons is otherwise specified, “lower alkyl”, as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. In certain embodiments, a substituent designated herein as alkyl is a lower alkyl.

The terms “alkoxyl” or “alkoxy” as used herein refers to an alkyl group, as defined below, having an oxygen moiety attached thereto. Representative alkoxyl groups include methoxy, ethoxy, propoxy, tert-butoxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₁, where m and R₁ are described below.

The terms “amine” and “amino” are art-recognized and refer to both unsubstituted and substituted amines, e.g., a moiety that can be represented by the general formulae:

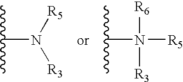

wherein R₃, R₅, and R₆ each independently represent a hydrogen, an alkyl, an alkenyl, —(CH₂)ₘ—R₁, or R₃ and R₅ taken together with the N atom to which they are attached complete a heterocycle having from 4 to 8 atoms in the ring structure; R₁ represents an alkenyl, aryl, cycloalkyl, a cycloalkenyl, a heterocyclyl, or a polycyclyl; and m is zero or an integer in the range of 1 to 8. In certain embodiments, only one of R₃ or R₅ can be a carbonyl, e.g., R₃, R₅, and the nitrogen together do not form an imide. In even more certain embodiments, R₃ and R₅ (and optionally R₆) each independently represent a hydrogen, an alkyl, an alkenyl, or —(CH₂)ₘ—R₁. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a conjugate acid with a pKa ≧7.00, i.e., the protonated forms of these functional groups have pKas relative to water above about 7.00.

The term “carbonyl” is art-recognized and includes such moieties as can be represented by the general formula:

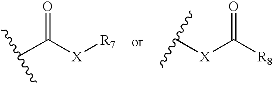

wherein X is a bond or represents an oxygen or a sulfur, and R₇ represents a hydrogen, an alkyl, an alkenyl, —(CH₂)ₘ—R₁ or a pharmaceutically acceptable salt, R₈ represents a hydrogen, an alkyl, an alkenyl or —(CH₂)ₘ—R₁, where m and R₁ are as defined above. Where X is an oxygen and R₇ or R₈ is not hydrogen, the formula represents an “ester.” Where X is an oxygen, and R₇ is as defined above, the moiety is referred to herein as a carboxyl group, and particularly when R₇ is a hydrogen, the formula represents a “carboxylic acid.” Where X is an oxygen, and R₈ is a hydrogen, the formula represents a “formate.” In general, where the oxygen atom of the above formula is replaced by a sulfur, the formula represents a “thiocarbonyl” group. Where X is a sulfur and R₇ or R₈ is not hydrogen, the formula represents a “thioester” group. Where X is a sulfur and R₇ is a hydrogen, the formula represents a “thiocarboxylic acid” group. Where X is a sulfur and R₈ is a hydrogen, the formula represents a “thioformate” group. On the other hand, where X is a bond, and R₇ is not hydrogen, the above formula represents a “ketone” group. Where X is a bond, and R₇ is a hydrogen, the above formula represents an “aldehyde” group.

The terms “heterocyclyl” or “heterocyclic group” refer to substituted or unsubstituted non-aromatic 3- to 10-membered ring structures, more preferably 3- to 7-membered rings, whose ring structures include one to four heteroatoms. The term terms “heterocyclyl” or “heterocyclic group” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As used herein, the definition of each expression, e.g., alkyl, m, n, etc., when it occurs more than once in any structure, is intended to be independent of its definition elsewhere in the same structure.

The terms “amino acid residue” and “peptide residue” mean an amino acid or peptide molecule without the —OH of its carboxyl group. In general the abbreviations used herein for designating the amino acids and the protective groups are based on recommendations of the IUPAC-IUB Commission on Biochemical Nomenclature (see Biochemistry 1972, 11, 1726-1732). For instance, Met, Ile, Leu, Ala, and Gly represent “residues” of methionine, isoleucine, leucine, alanine, and glycine, respectively. Residue means a moiety derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by K. D. Kopple, Peptides and Amino Acids; Benjamin: New York, 1966; pp. 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H-(the side chain of glycine).

In certain embodiments, the amino acids used in the present invention are those naturally occurring amino acids found in native proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs which have been identified as constituents of peptidylglycan bacterial cell walls.

The term amino acid residue further includes analogs, derivatives and congeners of any specific amino acid referred to herein, as well as C-terminal or N-terminal protected amino acid derivatives (e.g., modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated, the amino acid or residue can have the configuration (D), (L), or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) and (L) stereoisomers.

The term “peptide analogue,” as used herein, refers to a peptide comprising one or more non-naturally occurring amino acid. Examples of non-naturally occurring amino acids include, but are not limited to, D-amino acids (i.e., an amino acid of an opposite chirality to the naturally occurring form), N-α-methyl amino acids, C-α-methyl amino acids, β-methyl amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine, D- or L-p-methoxybiphenylalanine, methionine sulphoxide (MSO) and homoarginine (Har). Other examples include D- or L-2-indole(alkyl)alanines and D- or L-alkylalanines, wherein alkyl is substituted or unsubstituted methyl, ethyl, propyl, hexyl, butyl, pentyl, isopropyl, iso-butyl, or iso-pentyl, and phosphono- or sulfated (e.g., —SO₃H) non-carboxylate amino acids.

Other examples of non-naturally occurring amino acids include 3-(2-chlorophenyl)-alanine, 3-chloro-phenylalanine, 4-chloro-phenylalanine, 2-fluoro-phenylalanine, 3-fluoro-phenylalanine, 4-fluoro-phenylalanine, 2-bromo-phenylalanine, 3-bromo-phenylalanine, 4-bromo-phenylalanine, homophenylalanine, 2-methyl-phenylalanine, 3-methyl-phenylalanine, 4-methyl-phenylalanine, 2,4-dimethyl-phenylalanine, 2-nitro-phenylalanine, 3-nitro-phenylalanine, 4-nitro-phenylalanine, 2,4-dinitro-phenylalanine, 1,2,3,4-Tetrahydroisoquinoline-3-carboxylic acid, 1,2,3,4-tetrahydronorharman-3-carboxylic acid, 1-naphthylalanine, 2-naphthylalanine, pentafluorophenylalanine, 2,4-dichloro-phenylalanine, 3,4-dichloro-phenylalanine, 3,4-difluoro-phenylalanine, 3,5-difluoro-phenylalanine, 2,4,5-trifluoro-phenylalanine, 2-trifluoromethyl-phenylalanine, 3-trifluoromethyl-phenylalanine, 4-trifluoromethyl-phenylalanine, 2-cyano-phenyalanine, 3-cyano-phenyalanine, 4-cyano-phenyalanine, 2-iodo-phenyalanine, 3-iodo-phenyalanine, 4-iodo-phenyalanine, 4-methoxyphenylalanine, 2-aminomethyl-phenylalanine, 3-aminomethyl-phenylalanine, 4-aminomethyl-phenylalanine, 2-carbamoyl-phenylalanine, 3-carbamoyl-phenylalanine, 4-carbamoyl-phenylalanine, m-tyrosine, 4-amino-phenylalanine, styrylalanine, 2-amino-5-phenyl-pentanoic acid, 9-anthrylalanine, 4-tert-butyl-phenylalanine, 3,3-diphenylalanine, 4,4′-diphenylalanine, benzoylphenylalanine, α-methyl-phenylalanine, α-methyl-4-fluoro-phenylalanine, 4-thiazolylalanine, 3-benzothienylalanine, 2-thienylalanine, 2-(5-bromothienyl)-alanine, 3-thienylalanine, 2-furylalanine, 2-pyridylalanine, 3-pyridylalanine, 4-pyridylalanine, 2,3-diaminopropionic acid, 2,4-diaminobutyric acid, allylglycine, 2-amino-4-bromo-4-pentenoic acid, propargylglycine, 4-aminocyclopent-2-enecarboxylic acid, 3-aminocyclopentanecarboxylic acid, 7-amino-heptanoic acid, dipropylglycine, pipecolic acid, azetidine-3-carboxylic acid, cyclopropylglycine, cyclopropylalanine, 2-methoxy-phenylglycine, 2-thienylglycine, 3-thienylglycine, α-benzyl-proline, α-(2-fluoro-benzyl)-proline, α-(3-fluoro-benzyl)-proline, α-(4-fluoro-benzyl)-proline, α-(2-chloro-benzyl)-proline, α-(3-chloro-benzyl)-proline, α-(4-chloro-benzyl)-proline, α-(2-bromo-benzyl)-proline, α-(3-bromo-benzyl)-proline, α-(4-bromo-benzyl)-proline, α-phenethyl-proline, α-(2-methyl-benzyl)-proline, α-(3-methyl-benzyl)-proline, α-(4-methyl-benzyl)-proline, α-(2-nitro-benzyl)-proline, α-(3-nitro-benzyl)-proline, α-(4-nitro-benzyl)-proline, α-(1-naphthalenylmethyl)-proline, α-(2-naphthalenylmethyl)-proline, α-(2,4-dichloro-benzyl)-proline, α-(3,4-dichloro-benzyl)-proline, α-(3,4-difluoro-benzyl)-proline, α-(2-trifluoromethyl-benzyl)-proline, α-(3-trifluoromethyl-benzyl)-proline, α-(4-trifluoromethyl-benzyl)-proline, α-(2-cyano-benzyl)-proline, α-(3-cyano-benzyl)-proline, α-(4-cyano-benzyl)-proline, α-(2-iodo-benzyl)-proline, α-(3-iodo-benzyl)-proline, α-(4-iodo-benzyl)-proline, α-(3-phenyl-allyl)-proline, α-(3-phenyl-propyl)-proline, α-(4-tert-butyl-benzyl)-proline, α-benzhydryl-proline, α-(4-biphenylmethyl)-proline, α-(4-thiazolylmethyl)-proline, α-(3-benzo[b]thiophenylmethyl)-proline, α-(2-thiophenylmethyl)-proline, α-(5-bromo-2-thiophenylmethyl)-proline, α-(3-thiophenylmethyl)-proline, α-(2-furanylmethyl)-proline, α-(2-pyridinylmethyl)-proline, α-(3-pyridinylmethyl)-proline, α-(4-pyridinylmethyl)-proline, α-allyl-proline, α-propynyl-proline, γ-benzyl-proline, γ-(2-fluoro-benzyl)-proline, γ-(3-fluoro-benzyl)-proline, γ-(4-fluoro-benzyl)-proline, γ-(2-chloro-benzyl)-proline, γ-(3-chloro-benzyl)-proline, γ-(4-chloro-benzyl)-proline, γ-(2-bromo-benzyl)-proline, γ-(3-bromo-benzyl)-proline, γ-(4-bromo-benzyl)-proline, γ-(2-methyl-benzyl)-proline, γ-(3-methyl-benzyl)-proline, γ-(4-methyl-benzyl)-proline, γ-(2-nitro-benzyl)-proline, γ-(3-nitro-benzyl)-proline, γ-(4-nitro-benzyl)-proline, γ-(1-naphthalenylmethyl)-proline, γ-(2-naphthalenylmethyl)-proline, γ-(2,4-dichloro-benzyl)-proline, γ-(3,4-dichloro-benzyl)-proline, γ-(3,4-difluoro-benzyl)-proline, γ-(2-trifluoromethyl-benzyl)-proline, γ-(3-trifluoromethyl-benzyl)-proline, γ-(4-trifluoromethyl-benzyl)-proline, γ-(2-cyano-benzyl)-proline, γ-(3-cyano-benzyl)-proline, γ-(4-cyano-benzyl)-proline, γ-(2-iodo-benzyl)-proline, γ-(3-iodo-benzyl)-proline, γ-(4-iodo-benzyl)-proline, γ-(3-phenyl-allyl-benzyl)-proline, γ-(3-phenyl-propyl-benzyl)-proline, γ-(4-tert-butyl-benzyl)-proline, γ-benzhydryl-proline, γ-(4-biphenylmethyl)-proline, γ-(4-thiazolylmethyl)-proline, γ-(3-benzothioienylmethyl)-proline, γ-(2-thienylmethyl)-proline, γ-(3-thienylmethyl)-proline, γ-(2-furanylmethyl)-proline, γ-(2-pyridinylmethyl)-proline, γ-(3-pyridinylmethyl)-proline, γ-(4-pyridinylmethyl)-proline, γ-allyl-proline, γ-propynyl-proline, trans-4-phenyl-pyrrolidine-3-carboxylic acid, trans-4-(2-fluoro-phenyl)-pyrrolidine-3-carboxylic acid, trans-4-(3-fluoro-phenyl)-pyrrolidine-3-carboxylic acid, trans-4-(4-fluoro-phenyl)-pyrrolidine-3-carboxylic acid, trans-4-(2-chloro-phenyl)-pyrrolidine-3-carboxylic acid, trans-4-(3-chloro-phenyl)-pyrrolidine-3-carboxylic acid, trans-4-(4-chloro-phenyl)-pyrrolidine-3-carboxylic acid, trans-4-(2-bromo-phenyl)-pyrrolidine-3-carboxylic acid, trans-4-(3-bromo-phenyl)-pyrrolidine-3-carboxylic acid, trans-4-(4-bromo-phenyl)-pyrrolidine-3-carboxylic acid, trans-4-(2-methyl-phenyl)-pyrrolidine-3-carboxylic acid, trans-4-(3-methyl-phenyl)-pyrrolidine-3-carboxylic acid, trans-4-(4-methyl-phenyl)-pyrrolidine-3-carboxylic acid, trans-4-(2-nitro-phenyl)-pyrrolidine-3-carboxylic acid, trans-4-(3-nitro-phenyl)-pyrrolidine-3-carboxylic acid, trans-4-(4-nitro-phenyl)-pyrrolidine-3-carboxylic acid, trans-4-(1-naphthyl)-pyrrolidine-3-carboxylic acid, trans-4-(2-naphthyl)-pyrrolidine-3-carboxylic acid, trans-4-(2,5-dichloro-phenyl)-pyrrolidine-3-carboxylic acid, trans-4-(2,3-dichloro-phenyl)-pyrrolidine-3-carboxylic acid, trans-4-(2-trifluoromethyl-phenyl)-pyrrolidine-3-carboxylic acid, trans-4-(3-trifluoromethyl-phenyl)-pyrrolidine-3-carboxylic acid, trans-4-(4-trifluoromethyl-phenyl)-pyrrolidine-3-carboxylic acid, trans-4-(2-cyano-phenyl)-pyrrolidine-3-carboxylic acid, trans-4-(3-cyano-phenyl)-pyrrolidine-3-carboxylic acid, trans-4-(4-cyano-phenyl)-pyrrolidine-3-carboxylic acid, trans-4-(2-methoxy-phenyl)-pyrrolidine-3-carboxylic acid, trans-4-(3-methoxy-phenyl)-pyrrolidine-3-carboxylic acid, trans-4-(4-methoxy-phenyl)-pyrrolidine-3-carboxylic acid, trans-4-(2-hydroxy-phenyl)-pyrrolidine-3-carboxylic acid, trans-4-(3-hydroxy-phenyl)-pyrrolidine-3-carboxylic acid, trans-4-(4-hydroxy-phenyl)-pyrrolidine-3-carboxylic acid, trans-4-(2,3-dimethoxy-phenyl)-pyrrolidine-3-carboxylic acid, trans-4-(3,4-dimethoxy-phenyl)-pyrrolidine-3-carboxylic acid, trans-4-(3,5-dimethoxy-phenyl)-pyrrolidine-3-carboxylic acid, trans-4-(2-pyridinyl)-pyrrolidine-3-carboxylic acid, trans-4-(3-pyridinyl)-pyrrolidine-3-carboxylic acid, trans-4-(6-methoxy-3-pyridinyl)-pyrrolidine-3-carboxylic acid, trans-4-(4-pyridinyl)-pyrrolidine-3-carboxylic acid, trans-4-(2-thienyl)-pyrrolidine-3-carboxylic acid, trans-4-(3-thienyl)-pyrrolidine-3-carboxylic acid, trans-4-(2-furanyl)-pyrrolidine-3-carboxylic acid, trans-4-isopropyl-pyrrolidine-3-carboxylic acid, 4-phosphonomethyl-phenylalanine, benzyl-phosphothreonine, (1′-amino-2-phenyl-ethyl)oxirane, (1′-amino-2-cyclohexyl-ethyl)oxirane, (1′-amino-2-[3-bromo-phenyl]ethyl)oxirane, (1′-amino-2-[4-(benzyloxy)phenyl]ethyl)oxirane, (1′-amino-2-[3,5-difluoro-phenyl]ethyl)oxirane, (1′-amino-2-[4-carbamoyl-phenyl]ethyl)oxirane, (1′-amino-2-[benzyloxy-ethyl])oxirane, (1′-amino-2-[4-nitro-phenyl]ethyl)oxirane, (1′-amino-3-phenyl-propyl)oxirane, (1′-amino-3-phenyl-propyl)oxirane, and/or salts and/or protecting group variants thereof.

The term “peptide derivative,” as used herein, refers to a peptide comprising additional chemical or biochemical moieties not normally a part of a naturally occurring peptide. Peptide derivatives include peptides in which the amino-terminus and/or the carboxy-terminus and/or one or more amino acid side chain has been derivatised with a suitable chemical substituent group, as well as cyclic peptides, dual peptides, multimers of the peptides, peptides fused to other proteins or carriers, glycosylated peptides, phosphorylated peptides, peptides conjugated to lipophilic moieties (for example, caproyl, lauryl, stearoyl moieties) and peptides conjugated to an antibody or other biological ligand. Examples of chemical substituent groups that may be used to derivatise a peptide include, but are not limited to, alkyl, cycloalkyl and aryl groups; acyl groups, including alkanoyl and aroyl groups; esters; amides; halogens; hydroxyls; carbamyls, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides. The substituent group may be a “protecting group” as detailed herein.

The phrase “protecting group” as used herein means substituents which protect the reactive functional group from undesirable chemical reactions. Examples of such protecting groups include esters of carboxylic acids and boronic acids, ethers of alcohols, and acetals and ketals of aldehydes and ketones. For instance, the phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as, to illustrate, formyl, dansyl, acetyl, benzoyl, trifluoroacetyl, succinyl, and methoxysuccinyl; aromatic urethane protecting groups as, for example, benzyloxycarbonyl (Cbz); and aliphatic urethane protecting groups such as t-butoxycarbonyl (Boc) or 9-Fluorenylmethoxycarbonyl (Fmoc).

The term “amino-protecting group” or “N-terminal protecting group” refers to those groups intended to protect the α-N-terminal of an amino acid or peptide or to otherwise protect the amino group of an amino acid or peptide against undesirable reactions during synthetic procedures. Commonly used N-protecting groups are disclosed in Greene, Protective Groups In Organic Synthesis, (John Wiley & Sons, New York (1981)), which is hereby incorporated by reference. Additionally, protecting groups can be used as pro-drugs which are readily cleaved in vivo, for example, by enzymatic hydrolysis, to release the biologically active parent. α-N-Protecting groups comprise lower alkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyoxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like. Still other examples include theyl, succinyl, methoxysuccinyl, subery, adipyl, azelayl, dansyl, benzyloxycarbonyl, methoxyazelaly, methoxyadipyl, methoxysuberyl, and 2,4-dinitrophenyl.

The term “carboxy protecting group” or “C-terminal protecting group” refers to a carboxylic acid protecting ester or amide group employed to block or protect the carboxylic acid functionality while the reactions involving other functional sites of the compound are performed. Carboxy protecting groups are disclosed in Greene, Protective Groups in Organic Synthesis pp. 152-186 (1981), which is hereby incorporated by reference. Additionally, a carboxy protecting group can be used as a pro-drug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups are C₁-C₈ loweralkyl (e.g., methyl, ethyl or t-butyl and the like); arylalkyl such as phenethyl or benzyl and substituted derivatives thereof such as alkoxybenzyl or nitrobenzyl groups and the like; arylalkenyl such as phenylethenyl and the like; aryl and substituted derivatives thereof such as 5-indanyl and the like; dialkylaminoalkyl such as dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl such as benzoyloxymethyl, benzoyloxyethyl and the like; arylalkylcarbonyloxyalkyl such as benzylcarbonyloxymethyl, 2-benzylcarbonyloxyethyl and the like; alkoxycarbonylalkyl or cycloalkyloxycarbonylalkyl such as methoxycarbonylmethyl, cyclohexyloxycarbonylmethyl, 1-methoxycarbonyl-1-ethyl and the like; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl and the like; aryloxycarbonyloxyalkyl such as 2-(phenoxycarbonyloxy)ethyl, 2-(5-indanyloxycarbonyloxy)ethyl and the like; alkoxyalkylcarbonyloxyalkyl such as 2-(1-methoxy-2-methylpropan-2-oyloxy)ethyl and like; arylalkyloxycarbonyloxyalkyl such as 2-(benzyloxycarbonyloxy)ethyl and the like; arylalkenyloxycarbonyloxyalkyl such as 2-(3-phenylpropen-2-yloxycarbonyloxy)ethyl and the like; alkoxycarbonylaminoalkyl such as t-butyloxycarbonylaminomethyl and the like; alkylaminocarbonylaminoalkyl such as methylaminocarbonylaminomethyl and the like; alkanoylaminoalkyl such as acetylaminomethyl and the like; heterocycliccarbonyloxyalkyl such as 4-methylpiperazinylcarbonyloxymethyl and the like; dialkylaminocarbonylalkyl such as dimethylaminocarbonylmethyl, diethylaminocarbonylmethyl and the like; (5-(loweralkyl)-2-oxo-1,3-dioxolen-4-yl)alkyl such as (5-t-butyl-2-oxo-1,3-dioxolen-4-yl)methyl and the like; and (5-phenyl-2-oxo-1,3-dioxolen-4-yl)alkyl such as (5-phenyl-2-oxo-1,3-dioxolen-4-yl)methyl and the like. Representative amide carboxy protecting groups are aminocarbonyl and loweralkylaminocarbonyl groups. For example, aspartic acid may be protected at the α-C-terminal by an acid labile group (e.g., t-butyl) and protected at the β-C-terminal by a hydrogenation labile group (e.g., benzyl) then deprotected selectively during synthesis. As mentioned above, the protected carboxy group may also be a loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester.

As noted above, certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

For purposes of this invention, the chemical elements are identified in accordance with the Periodic Table of the Elements, CAS version, Handbook of Chemistry and Physics, 67th ed., 1986-87, inside cover.

A compound is said to have an “insulinotropic activity” if it is able to stimulate, or cause the stimulation of, the synthesis or expression of the hormone insulin.

Another aspect of the present invention relates to pharmaceutical compositions of dipeptidylpeptidase inhibitors, particularly inhibitor(s) and their uses in treating and/or preventing disorders which can be improved by altering the homeostasis of peptide hormones. In an embodiment, the inhibitors have hypoglycemic and antidiabetic activities, and can be used in the treatment of disorders marked by aberrant glucose metabolism (including storage). In particular embodiments, the compositions of the subject methods are useful as insulinotropic agents, or to potentiate the insulinotropic effects of such molecules as GLP-1. In this regard, the present method can be useful for the treatment and/or prophylaxis of a variety of disorders, including one or more of: hyperlipemia, hyperglycemia, obesity, glucose tolerance insufficiency, insulin resistance, and diabetic complications.

For instance, in certain embodiments the method involves administration of an inhibitor(s), preferably at a predetermined interval(s) during a 24-hour period, in an amount effective to improve one or more aberrant indices associated with glucose metabolism disorders (e.g., glucose intolerance, insulin resistance, hyperglycemia, hyperinsulinemia, and Type II diabetes). The effective amount of the inhibitor may be about 0.01, 0.1, 1, 10, 30, 50, 70, 100, 150, 200, 500, or 1000 mg/kg of the subject.

### (ii) Agonism of GLP-1 Effects

The compounds useful in the subject methods possess, in certain embodiments, the ability to lower blood glucose levels, to relieve obesity, to alleviate impaired glucose tolerance, to inhibit hepatic glucose neogenesis, and to lower blood lipid levels and to inhibit aldose reductase. They are thus useful for the prevention and/or therapy of hyperglycemia, obesity, hyperlipidemia, diabetic complications (including retinopathy, nephropathy, neuropathy, cataracts, coronary artery disease and arteriosclerosis), and furthermore for obesity-related hypertension and osteoporosis.

Diabetes mellitus is a disease characterized by hyperglycemia caused by from a decrease in insulin secretion, decreased insulin sensitivity, and/or insulin resistance. The morbidity and mortality of this disease result from vascular, renal, and neurological complications. An oral glucose tolerance test is a clinical test used to diagnose diabetes. In an oral glucose tolerance test, a patient's physiological response to a glucose load or challenge is evaluated. After ingesting the glucose, the patient's physiological response to the glucose challenge is evaluated. Generally, this is accomplished by determining the patient's blood glucose levels (the concentration of glucose in the patient's plasma, serum, or whole blood) at several predetermined points in time.

In one embodiment, the present invention provides a method for agonizing the action of GLP-1. It has been determined that isoforms of GLP-1 (GLP-1(7-37) and GLP-1(7-36)), which are derived from preproglucagon in the intestine and the hind brain, have insulinotropic activity; i.e., they modulate glucose metabolism. DPIV cleaves the isoforms to inactive peptides. Thus, in certain embodiments, compound(s) of the present invention can agonize insulinotropic activity by interfering with the degradation of bioactive GLP-1 peptides.

### (iii) Agonism of the Effects of Other Peptide Hormones

In another embodiment, the subject agents can be used to agonize (e.g., mimic or potentiate) the activity of peptide hormones, e.g., GLP-2, GIP and NPY.

To illustrate further, the present invention provides a method for agonizing the action of GLP-2. It has been determined that GLP-2 acts as a trophic agent, to promote growth of gastrointestinal tissue. The effect of GLP-2 is marked particularly by increased growth of the small bowel, and is therefore herein referred to as an “intestinotrophic” effect. DPIV is known to cleave GLP-2 into a biologically inactive peptide. Thus, in one embodiment, inhibition of DPIV interferes with the degradation of GLP-2, and thereby increases the plasma half-life of that hormone.

In still other embodiments, the subject method can be used to increase the half-life of other proglucagon-derived peptides, such as glicentin, oxyntomodulin, glicentin-related pancreatic polypeptide (GRPP), and/or intervening peptide-2 (IP-2). For example, glicentin has been demonstrated to cause proliferation of intestinal mucosa and also inhibits a peristalsis of the stomach, and has thus been elucidated as useful as a therapeutic agent for digestive tract diseases.

Thus, in one aspect, the present invention relates to therapeutic and related uses of compound(s) for promoting the growth and proliferation of gastrointestinal tissue, particularly small bowel tissue. For instance, the subject method can be used as part of a regimen for treating injury, inflammation, or resection of intestinal tissue, e.g., where enhanced growth and repair of the intestinal mucosal epithelial is desired.

With respect to small bowel tissue, such growth is measured conveniently as an increase in small bowel mass and length, relative to an untreated control. The effect of compounds on small bowel tissue also manifests as an increase in the height of the crypt plus villus axis. Such activity is referred to herein as an “intestinotrophic” activity. The efficacy of the subject method may also be detectable as an increase in crypt cell proliferation and/or a decrease in small bowel epithelium apoptosis. These cellular effects may be noted most significantly in relation to the jejunum, including the distal jejunum and particularly the proximal jejunum, and also in the distal ileum. A compound is considered to have “intestinotrophic effect” if a test animal exhibits increased small bowel weight, increased height of the crypt plus villus axis or increased crypt cell proliferation, or decreased small bowel epithelium apoptosis when treated with the compound (or genetically engineered to express it themselves). A model suitable for determining such gastrointestinal growth is described by U.S. Pat. No. 5,834,428 (incorporated by reference).

In general, patients who would benefit from either increased small intestinal mass and consequent increased small bowel mucosal function are candidates for treatment by the subject method. Particular conditions that may be treated include the various forms of sprue, including celiac sprue which results from a toxic reaction to α-gliadin from wheat, and is marked by a tremendous loss of villae of the bowel; tropical sprue which results from infection and is marked by partial flattening of the villae; hypogammaglobulinemic sprue which is observed in patients with common variable immunodeficiency or hypogammaglobulinemia and is marked by significant decrease in villus height. The therapeutic efficacy of the treatment may be monitored by enteric biopsy to examine the villus morphology, by biochemical assessment of nutrient absorption, by patient weight gain, or by amelioration of the symptoms associated with these conditions. Other conditions that may be treated by the subject method, or for which the subject method may be useful prophylactically, include radiation enteritis, infectious or post-infectious enteritis, regional enteritis (Crohn's disease), small intestinal damage due to toxic or other chemotherapeutic agents, and patients with short bowel syndrome.

More generally, the present invention provides a therapeutic method for treating digestive tract diseases. The term “digestive tract” as used herein means a organismic tube through which food passes, including stomach and intestine. The term “digestive tract diseases” as used herein means diseases accompanied by a qualitative or quantitative abnormality in the digestive tract mucosa, which include ulceric or inflammatory disease; congenital or acquired digestion and absorption disorder including malabsorption syndrome; disease caused by loss of a mucosal barrier function of the gut; and protein-losing gastroenteropathy. The ulceric disease includes gastric ulcer, duodenal ulcer, small intestinal ulcer, colonic ulcer, and rectal ulcer. The inflammatory disease includes esophagitis, gastritis, duodenitis, enteritis, colitis, Crohn's disease, proctitis, gastrointestinal Behcet, radiation enteritis, radiation colitis, radiation proctitis, enteritis, and medicamentosa. The malabsorption syndrome includes the essential malabsorption syndrome, such as disaccharide-decomposing enzyme deficiency, glucose-galactose malabsorption, fructose malabsorption; secondary malabsorption syndrome, e.g., the disorder caused by a mucosal atrophy in the digestive tract through the intravenous or parenteral nutrition or elemental diet, the disease caused by the resection and shunt of the small intestine such as short gut syndrome, cul-de-sac syndrome; and indigestible malabsorption syndrome, such as the disease caused by resection of the stomach, e.g., dumping syndrome.

The term “therapeutic agent for digestive tract diseases” as used herein means the agents for the prevention and treatment of the digestive tract diseases, which include the therapeutic agent for digestive tract ulcer, the therapeutic agent for inflammatory digestive tract disease, the therapeutic agent for mucosal atrophy in the digestive tract, the therapeutic agent for a digestive tract wound, the amelioration agent for the function of the digestive tract including the agent for recovery of the mucosal barrier function, and the amelioration agent for digestive and absorptive function. Ulcers include digestive ulcers and erosions, and acute ulcers, namely acute mucosal lesions.

The subject method, because of promoting proliferation of intestinal mucosa, can be used in the treatment and prevention of pathologic conditions of insufficiency in digestion and absorption, that is, treatment and prevention of mucosal atrophy, or treatment of hypoplasia of the digestive tract tissues and decrease in these tissues by surgical removal as well as improvement of digestion and absorption. Further, the subject method can be used in the treatment of pathologic mucosal conditions due to inflammatory diseases, such as enteritis, Crohn's disease, and ulceric colitis and also in the treatment of reduction in function of the digestive tract after operation, for example, in damping syndrome as well as in the treatment of duodenal ulcer in conjunction with the inhibition of peristalsis of the stomach and rapid migration of food from the stomach to the jejunum. Furthermore, glicentin can effectively be used in promoting cure of surgical invasion as well as in improving functions of the digestive tract. Thus, the present invention also provides a therapeutic agent for atrophy of the digestive tract mucosa, a therapeutic agent for wounds in the digestive tract and a drug for improving functions of the digestive tract which comprise glicentin as active ingredients.

Likewise, the compound(s) of the subject invention can be used to alter the plasma half-life of secretin, VIP, PHI, PACAP, GIP, and/or helodermin. Additionally, the subject method can be used to alter the pharmacokinetics of Peptide YY and neuropeptide Y, both members of the pancreatic polypeptide family, as DPIV has been implicated in the processing of those peptides in a manner which alters receptor selectivity.

Neuropeptide Y (NPY) is believed to act in the regulation vascular smooth muscle tone, as well as regulation of blood pressure. NPY also decreases cardiac contractility. NPY is also the most powerful appetite stimulant known (Wilding et al., J. Endocrinology 1992, 132, 299-302). The centrally evoked food intake (appetite stimulation) effect is predominantly mediated by NPY Y1 receptors and causes increase in body fat stores and obesity (Stanley et al., Physiology and Behavior 1989, 46, 173-177).

According to the present invention, a method for treatment of anorexia comprises administering to a subject in need thereof a therapeutically effective amount of a compound(s) of the present invention, to stimulate the appetite and increase body fat stores.

A method for treatment of hypotension comprises administering to a subject in need thereof a therapeutically effective amount of a compound(s) of the present invention to mediate vasoconstriction and increase blood pressure.

DPIV has also been implicated in the metabolism and inactivation of growth hormone-releasing factor (GHRF). GHRF is a member of the family of homologous peptides that includes glucagon, secretin, vasoactive intestinal peptide (VIP), peptide histidine isoleucine (PHI), pituitary adenylate cyclase activating peptide (PACAP), gastric inhibitory peptide (GIP) and helodermin (Kubiak et al. Peptide Res. 1994, 7, 153). GHRF is secreted by the hypothalamus, and stimulates the release of growth hormone (GH) from the anterior pituitary. Thus, the subject method can be used to improve clinical therapy for certain growth hormone deficient children, and in clinical therapy of adults to improve nutrition and to alter body composition (muscle vs. fat). The subject method can also be used in veterinary practice, for example, to develop higher yield milk production and higher yield, leaner livestock.

### (iv) Assays of Insulinotropic Activity

In selecting a compound suitable for use in the subject method, it is noted that the insulinotropic property of a compound may be determined by providing that compound to animal cells, or injecting that compound into animals and monitoring the release of immunoreactive insulin (IRI) into the media or circulatory system of the animal, respectively. The presence of IRI can be detected through the use of a radioimmunoassay which can specifically detect insulin.

The db/db mouse is a genetically obese and diabetic strain of mouse. The db/db mouse develops hyperglycemia and hyperinsulinemia concomitant with its development of obesity and thus serves as a model of obese type 2 diabetes (NIDDM). The db/db mice can be purchased from, for example, The Jackson Laboratories (Bar Harbor, Me.). In an exemplary embodiment, for treatment of the mice with a regimen including a compound(s) or control, sub-orbital sinus blood samples are taken before and at some time (e.g., 60 min) after dosing of each animal. Blood glucose measurements can be made by any of several conventional techniques, such as using a glucose meter. The blood glucose levels of the control and compound(s) dosed animals are compared.

The metabolic fate of exogenous GLP-1 can also be followed in both nondiabetic and type II diabetic subjects, and the effect of a candidate compound(s) determined. For instance, a combination of high-pressure liquid chromatography (HPLC), specific radioimmunoassays (RIAs), and an enzyme-linked immunosorbent assay (ELISA), can be used, whereby intact biologically active GLP-1 and its metabolites can be detected. See, for example, Deacon et al. Diabetes, 1995, 44, 1126-1131. To illustrate, after GLP-1 administration, the intact peptide can be measured using an NH₂-terminally directed RIA or ELISA, while the difference in concentration between these assays and a COOH-terminal-specific RIA allowed determination of NH₂-terminally truncated metabolites. Without compound, subcutaneous GLP-1 is rapidly degraded in a time-dependent manner, forming a metabolite which co-elutes on HPLC with GLP-1(9-36) amide and has the same immunoreactive profile. For instance, 30 min after subcutaneous GLP-1 administration to diabetic patients (n=8), the metabolite accounted for 88.5+1.9% of the increase in plasma immunoreactivity determined by the COOH-terminal RIA, which was higher than the levels measured in healthy subjects (78.4+3.2%; n=8; P<0.05). See Deacon et al., supra. Intravenously infused GLP-I was also extensively degraded.

### (v) Conjoint Administration

Another aspect of the invention provides a conjoint therapy wherein one or more other therapeutic agents are administered with the compound. Such conjoint treatment may be achieved by way of the simultaneous, sequential, or separate dosing of the individual components of the treatment.

In one embodiment, a compound(s) of the present invention is conjointly administered with insulin or other insulinotropic agents, such as GLP-1, peptide hormones, such as GLP-2, GIP, or NPY, or a gene therapy vector which causes the ectopic expression of said agents and peptide hormones. In certain embodiments, said agents or peptide hormones may be variants or derivatives of a naturally occurring or synthetic peptide hormone, wherein one or more amino acids have been added, deleted, or substituted.

In another illustrative embodiment, the compounds of the present invention can be conjointly administered with an M1 receptor antagonist. Cholinergic agents are potent modulators of insulin release that act via muscarinic receptors. Moreover, the use of such agents can have the added benefit of decreasing cholesterol levels, while increasing HDL levels. Suitable muscarinic receptor antagonists include substances that directly or indirectly block activation of muscarinic cholinergic receptors. Preferably, such substances are selective (or are used in amounts that promote such selectivity) for the M1 receptor. Non-limiting examples include quaternary amines (such as methantheline, ipratropium, and propantheline), tertiary amines (e.g., dicyclomine and scopolamine), and tricyclic amines (e.g., telenzepine). Other suitable muscarinic receptor antagonists include benztropine (commercially available as COGENTIN from Merck), hexahydro-sila-difenidol hydrochloride (HHSID hydrochloride disclosed in Lambrecht et al. Trends in Pharmacol. Sci. 1989, 10(Suppl), 60; (+/−)-3-quinuclidinyl xanthene-9-carboxylate hemioxalate (QNX-hemioxalate; Birdsall et al., Trends in Pharmacol. Sci. 1983, 4, 459; telenzepine dihydrochloride (Coruzzi et al. Arch. Int. Pharmacodyn. Ther. 1989, 302, 232; and Kawashima et al. Gen. Pharmacol. 1990, 21, 17), and atropine. The dosages of such muscarinic receptor antagonists will be generally subject to optimization as outlined above. In the case of lipid metabolism disorders, dosage optimization may be necessary independent of whether administration is timed by reference to the lipid metabolism responsiveness window or not.

In terms of regulating insulin and lipid metabolism and reducing the foregoing disorders, the compound(s) of the present invention may also act synergistically with prolactin inhibitors such as d2 dopamine agonists (e.g., bromocriptine). Accordingly, the subject method can include the conjoint administration of such prolactin inhibitors as prolactin-inhibiting ergo alkaloids and prolactin-inhibiting dopamine agonists. Examples of suitable compounds include 2-bromo-alpha-ergocriptine, 6-methyl-8-beta-carbobenzyloxyaminoethyl-10-alpha-ergoline, 8-acylaminoergolines, 6-methyl-8-alpha-(N-acyl)amino-9-ergoline, 6-methyl-8-alpha-(N-phenylacetyl)amino-9-ergoline, ergocornine, 9,10-dihydroergocornine, D-2-halo-6-alkyl-8-substituted ergolines, D-2-bromo-6-methyl-8-cyanomethylergoline, carbidopa, benserazide, and other dopadecarboxylase inhibitors, L-dopa, dopamine, and non toxic salts thereof.

The compound(s) of the present invention used according to the invention can also be used conjointly with agents acting on the ATP-dependent potassium channel of the β-cells, such as glibenclamide, glipizide, gliclazide, and AG-EE 623 ZW. The compound(s) may also advantageously be applied in combination with other oral agents, such as metformin and related compounds or glucosidase inhibitors, such as acarbose.

### (vi) Pharmaceutical Compositions

While it is possible for a compound of the present invention to be administered alone, in certain cases it is preferable to administer the compound as a pharmaceutical formulation (composition). Protease inhibitors according to the invention may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

Compounds prepared as described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means, and, if desired, the active ingredient may be mixed with any conventional additive, such as an excipient, a binder, a disintegrating agent, a lubricant, a corrigent, a solubilizing agent, a suspension aid, an emulsifying agent, or a coating agent. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses.

The precise time of administration and/or amount of the compound that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, e.g., Berge et al. J. Pharm. Sci. 1977, 66, 1-19)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, e.g., Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound(s) with the carrier and, optionally, one or more accessory ingredients. In general, the formulations are prepared by uniformly and intimately bringing into association a ligand with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes, and the like, each containing a predetermined amount of a compound(s) as an active ingredient. A compound may also be administered as a bolus, electuary or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more compound(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of a compound(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to compound(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The compound(s) can be alternatively administered by aerosol. This is accomplished by preparing an aqueous aerosol, liposomal preparation, or solid particles containing the compound. A non-aqueous (e.g., fluorocarbon propellant) suspension could be used.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Medicaments which may be administered in inhalant or aerosol formulations according to the invention include protease inhibitor prodrugs useful in inhalation therapy which may be presented in a form which is soluble or substantially soluble in the selected propellant system.

The particle size of the particulate medicament should be such as to permit inhalation of substantially all of the medicament into the lungs upon administration of the aerosol formulation and will thus desirably be less than 20 microns, preferably in the range 1 to 10 microns, e.g., 1 to 5 microns. The particle size of the medicament may be reduced by conventional means, for example by milling or micronisation.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1, 2, 3 or 4 puffs each time. Preferably, administration may be one time per day.

For administration, the drug is suitably inhaled from a nebulizer, from a pressurized metered dose inhaler, or as a dry powder from a dry powder inhaler (e.g., sold as TURBUHALER®) or from a dry powder inhaler utilizing gelatin, plastic or other capsules, cartridges or blister packs.

A diluent or carrier, generally non-toxic and chemically inert to the medicament; e.g., lactose, dextran, mannitol, glucose or any additives that will give the medicament a desired taste, can be added to the powdered medicament.

The micronized mixture may be suspended or dissolved in a liquid propellant mixture which is kept in a container that is sealed with a metering valve and fitted into a plastic actuator. The propellants used may be halocarbons of different chemical formulae. The most frequently used halocarbon propellants are trichlorofluoromethane, dichlorodifluoromethane, dichlorotetrafluoroethane, tetrafluoroethane, and 1,1-difluoroethane. Low concentrations of a surfactant such as sorbitan trioleate, lecithin, disodium dioctylsulphosuccinate, or oleic acid may also be used to improve the physical stability.

Transdermal patches have the added advantage of providing controlled delivery of a compound(s) to the body. Such dosage forms can be made by dissolving or dispersing the agent in the proper medium. Absorption enhancers can also be used to increase the flux of the compound(s) across the skin. The rate of such flux can be controlled by either providing a rate controlling membrane or dispersing the peptidomimetic in a polymer matrix or gel.

Ophthalmic formulations, eye ointments, powders, solutions, and the like, are also contemplated as being within the scope of this invention.

Pharmaceutical compositions of this invention suitable for parenteral administration comprise one or more compounds(s) in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of compound(s) in biodegradable polymers such as polylactide-polyglycolide. Depending on the ratio of drug to polymer, and the nature of the particular polymer employed, the rate of drug release can be controlled. Examples of other biodegradable polymers include poly-(orthoesters) and poly-(anhydrides). Depot injectable formulations are also prepared by entrapping the drug in liposomes or microemulsions which are compatible with body tissue.

When the compounds(s) of the present invention are administered as pharmaceuticals to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of agents may be given orally, parenterally, topically, or rectally. They are of course given by forms suitable for each administration route. For example, they are administered in tablets or capsule form, by injection, inhalation, eye lotion, ointment, suppository, infusion; topically by lotion or ointment; and rectally by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

These compounds(s) may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as Applied Animal Nutrition; San Francisco: Freedman, 1969; or Livestock Feeds and Feeding; Corvallis: O & B Books, 1977).

Regardless of the route of administration selected, the compound(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

### (vii) Combinatorial Libraries

The compounds of the present invention, particularly libraries of variants having various representative classes of substituents, are amenable to combinatorial chemistry and other parallel synthesis schemes (see, for example, PCT WO 94/08051). The result is that large libraries of related compounds, e.g., a variegated library of compounds represented above, can be screened rapidly in high throughput assays in order to identify potential protease inhibitor lead compounds, as well as to refine the specificity, toxicity, and/or cytotoxic-kinetic profile of a lead compound.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

Diversity in the library can be created at a variety of different levels. For instance, the substrate aryl groups used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules such as the subject protease inhibitors. See, for example, Blondelle et al. Trends Anal. Chem. 1995, 14, 83; the Affymax U.S. Pat. Nos. 5,359,115 and 5,362,899: the Ellman U.S. Pat. No. 5,288,514: the Still et al. PCT publication WO 94/08051; the ArQule U.S. Pat. Nos. 5,736,412 and 5,712,171; Chen et al. J. Am. Chem. Soc. 1994, 116, 2661: Kerr et al. J. Am. Chem. Soc. 1993, 115, 252; PCT publications WO92/10092, WO93/09668 and WO91/07087; and the Lerner et al. PCT publication WO93/20242). Accordingly, a variety of libraries on the order of about 100 to 1,000,000 or more diversomers of the subject protease inhibitors can be synthesized and screened for particular activity or property.

In an exemplary embodiment, a library of candidate protease inhibitor diversomers can be synthesized utilizing a scheme adapted to the techniques described in the Still et al. PCT publication WO 94/08051, e.g., being linked to a polymer bead by a hydrolyzable or photolyzable group, optionally located at one of the positions of the candidate agonists or a substituent of a synthetic intermediate. According to the Still et al. technique, the library is synthesized on a set of beads, each bead including a set of tags identifying the particular diversomer on that bead. The bead library can then be “plated” with proteases for which an inhibitor is sought. The diversomers can be released from the bead, e.g., by hydrolysis.

The structures of the compounds useful in the present invention lend themselves readily to efficient synthesis. The nature of the structures of the subject compounds, as generally set forth above, allows the rapid combinatorial assembly of such compounds. For example, as in the scheme set forth below, an activated aryl group, such as an aryl triflate or bromide, attached to a bead or other solid support can be linked to another aryl group by performing a Stille or Suzuki coupling with an aryl stannane or an aryl boronic acid. If the second aryl group is functionalized with an aldehyde, an amine substituent can be added through a reductive amination. Alternatively, the second aryl group could be functionalized with a leaving group, such as a triflate, tosylate, or halide, capable of being displaced by an amine. Or, the second aryl group may be functionalized with an amine group capable of undergoing reductive amination with an amine, e.g., CyKNH₂. Other possible coupling techniques include transition metal-mediated amine arylation reactions. The resultant secondary amine can then be further functionalized by an acylation, alkylation, or arylation to generate a tertiary amine or amide which can then be cleaved from the resin or support. These reactions generally are quite mild and have been successfully applied in combinatorial solid-phase synthesis schemes. Furthermore, the wide range of substrates and coupling partners suitable and available for these reactions permits the rapid assembly of large, diverse libraries of compounds for testing in assays as set forth herein. For certain schemes, and for certain substitutions on the various substituents of the subject compounds, one of skill in the art will recognize the need for masking certain functional groups with a suitable protecting group. Such techniques are well known in the art and are easily applied to combinatorial synthesis schemes.

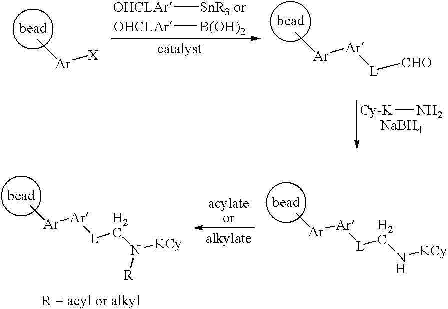

Many variations on the above and related pathways permit the synthesis of widely diverse libraries of compounds which may be tested as protease inhibitors.

Variations, modifications, and other implementations of what is described herein will occur to those of ordinary skill without departing from the spirit and the scope of the invention. Accordingly, the invention is not to be limited only to the preceding illustrative description. For additional illustrative features that may be used with the invention, including the embodiments described here, refer to the documents listed herein above and incorporated by reference in their entirety. All operative combinations between the above described illustrative embodiments and those features described below are considered to be potentially patentable embodiments of the invention.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Xaa-N(Methyl)-boroGly Inhibition of DPIV

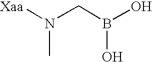

| | IC₅₀ (pH 2) | IC₅₀ (pH 8) | Cyclization Index |
|---|---|---|---|
| Xaa | nM | nM | (CI) |
| Trp | <50 | <300 | <50 |
| Pro | <50 | <30,000 | <1500 |
| Glu | <50 | <3,000 | <100 |
| Gly | <50 | <50,000 | <2000 |
| Val | <50 | <600 | <20 |
| Aad | <100 | <2,500 | <50 |
| Arg | <100 | <5,000 | <100 |
| 1-Naphthyl-Ala | <100 | <500 | <5 |
| Chg | <100 | <700 | <10 |
| (Cyclohexylglycine) | | | |
| Ile | <150 | <2,000 | <50 |
| t-Leu | <150 | <150 | <5 |
| Ethyl-Gly | <150 | <40,000 | <200 |
| Phe | <200 | <2,000 | <50 |
| Lys | <200 | <15,000 | <100 |
| Met | <400 | <25,000 | <100 |
| Tyr | <400 | <5,000 | <50 |
| Ala | <400 | <50,000 | <150 |
| n-Propyl-Gly | <400 | <50,000 | <100 |
| Thr | <700 | <50,000 | <100 |
| Leu | <1,000 | <75,000 | <100 |
| Gln | <1,000 | <50,000 | <50 |
| Ser | <1,500 | <25,000 | <200 |
| Asn | <2,500 | <75,000 | <500 |
| Asp | <35,000 | <300,000 | <10 |
| His | <50,000 | <150,000 | <10 |
| Methyl-Gly | <10,000 | <1,500,000 | <150 |
| Ethyl-Gly | <10,000 | <1,500,000 | <200 |
| t-Butyl-Gly | <150 | <150 | <1 |
| Methyl-Ala | <75,000 | <1,000,000 | <5 |
| Aib | <210,000 | N/A | N/A |
| N-Methyl-Gly | <10,000 | <1,500,000 | <150 |
| N-Ethyl-Gly | <10,000 | <1,500,000 | <200 |
| N-t-Butyl-Gly | <150 | <150 | <1 |
| N-Methyl-Ala | <75,000 | <1,000,000 | <5 |

The cyclization index (CI) as used herein is the ratio of the IC₅₀ at pH 8 and the IC₅₀ at pH 2, wherein the greater the value of CI, the more the equilibrium favors the cyclized species. While not intending to be bound by a particular mechanism, it has been proposed that the cyclization process may occur through a mechanism as shown in Scheme 1.

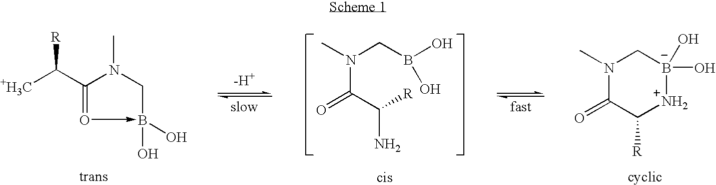

### Example 2

### Xaa-N(Ethyl)-boroGly Inhibition of DPIV

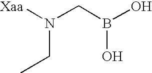

| | IC₅₀ (pH 2) | IC₅₀ (pH 8) | Cyclization Index |
|---|---|---|---|
| Xaa | nM | nM | (CI) |
| Trp | <50 | <50 | <1 |
| Pro | <100 | <20,000 | <300 |
| Glu | <100 | <200 | <5 |
| Gly | <1,500 | <300,000 | <200 |
| Val | <100 | <100 | <5 |
| Aad | <100 | <300 | <5 |
| Arg | <200 | <500 | <5 |
| 1-Naphthyl-Ala | <200 | <200 | <1 |
| Chg | <250 | <200 | <1 |
| (Cyclohexylglycine) | | | |
| Ile | <200 | <200 | <5 |
| t-Leu | <400 | <200 | <1 |
| Ethyl-Gly | <300 | <500 | <5 |
| Phe | <300 | <300 | <5 |
| Lys | <250 | <500 | <5 |
| Tyr | <400 | <500 | <5 |
| Ala | <500 | <1,500 | <5 |
| n-Propyl-Gly | <500 | <1,000 | <5 |
| Leu | <1,000 | <3,000 | <5 |
| Gln | <5,000 | <10,000 | <5 |
| Ser | <1,500 | <10,000 | <5 |
| Asn | <10,000 | <50,000 | <10 |
| Asp | <10,000 | <30,000 | <5 |
| His | <20,000 | <50,000 | <5 |
| Aib | <500,000 | <500,000 | <5 |
| N-Methyl-Gly | <10,000 | <1,500,000 | <150 |
| N-Ethyl-Gly | <10,000 | <1,500,000 | <200 |

### Example 3

### General Procedure for the Preparation of Inhibitors

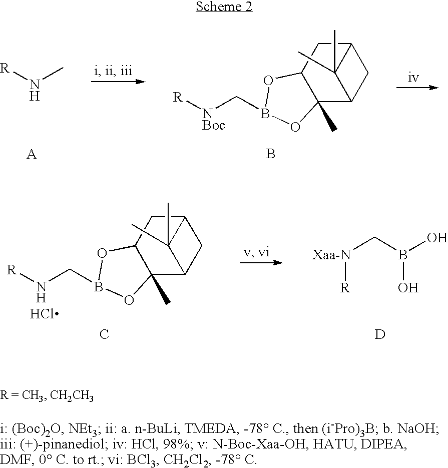

(+)-Pinanediol N-Boc-borosarcosine (B, R═CH₃). N-Boc-dimethylamine was prepared from dimethylamine using the general method (Yield: 90%). N-Boc-dimethylamine (14.5 g, 100 mmol) was dissolved in a mixture of Et₂O (200 mL) and TMEDA (30 mL, 200 mmol) and cooled to −78° C. A solution of s-BuLi (92 mL of a 1.3 M solution in cyclohexane, 120 mmol) was added. The reaction mixture was stirred for 3 h at −78° C. and treated with (i-PrO)₃B (56.4 g, 300 mmol). The cooling bath was then removed and the solution allowed to warm to room temperature, quenched by the addition of H₂O and extracted into 2N NaOH (2×100 mL). The aqueous phase was acidified to pH 3 using 2 N HCl and extracted with EtOAc. The extracts were dried over MgSO₄ and concentrated to produce crude N-Boc-boroSar which was redissolved in Et₂O (200 mL), added (+)-pinanediol (17.0 g, 100 mmol) and stirred for 2 h at room temperature. The resulting mixture was then dried over MgSO₄, filtered, concentrated and purified by flash column chromatography (3:1, hexanes/EtOAc) to afford B (R═CH₃) (17.2 g, 58%) as a colorless oil. ¹HNMR (CDCl₃): δ 0.85 (s, 3H, pinanyl CH₃), 1.28 (s, 3H, pinanyl CH₃), 1.39 (s, 3H, pinanyl CH₃), 1.47 (s, 9H, 3×CH₃ of Boc), 1.82-2.32 (m, 5H, pinanyl protons), 2.59 (br s, 2H, NCH₂B), 2.86 (s, 3H, NCH₃), 4.26 (d, J=8.7 Hz, 1H, pinanyl BOCH).

(+)-Pinanediol borosarcosine (C, R═CH₃). Compound B (R═CH₃) (14.8 g, 50 mmol) was dissolved in a 4 M solution of HCl in dioxane (40 mL) and stirred for 2 h at room temperature. The solvent was dried to afford compound C(R═CH₃) (11.5 g, 98%) as a white powder. ¹HNMR (CDCl₃): δ 0.83 (s, 3H, pinanyl CH₃), 1.19 (d, J=11.0 Hz, 1H, pinanyl H_{endo})_{,} 1.28 (s, 3H, pinanyl CH₃), 1.44 (s, 3H, pinanyl CH₃), 1.92-2.29 (m, 5H, pinanyl protons), 2.69 (s, 2H, NCH₂B), 2.77 (s, 3H, NCH₃), 4.40 (dd, J=8.7, 2.0 Hz, 1H, pinanyl BOCH), 9.39 (br s, 2H, ⁺NH₂).

(+)-Pinanediol N-EthylboroGlycine (C, R═CH₂CH₃). Starting from N-Boc-N-methylethylamine followed the same procedure as described above compound C (R═CH₂CH₃) was obtained as a white powder. ¹HNMR (CDCl₃): δ 0.83 (s, 3H, pinanyl CH₃), 1.20 (d, J=11.0 Hz, 1H, pinanyl H_{endo}), 1.28 (s, 3H, pinanyl CH₃), 1.44-1.48 (m, 6H, pinanyl CH₃, CH₃CH₂), 1.93-2.36 (m, 5H, pinanyl protons), 2.67 (s, 2H, NCH₂B), 3.15 (d, 2H, NCH₂CH₃), 4.41 (dd, J=8.7, 2.0 Hz, 1H, pinanyl BOCH), 9.40 (br s, 2H, ⁺NH₂).

General Procedure for the Synthesis of Dipeptide Sarcosine Boronic Acids (D, R═CH₃). To a stirred mixture of N-protected amino acid (0.5 mmol), C (130 mg, 0.5 mmol), HATU (200 mg, 0.526 mmol), and anhydrous DMF (2 mL) was added DIPEA (142 mg, 1.1 mmol) under Ar at 0° C., after 15 min the reaction mixture was warmed to room temperature and stirred overnight. The reaction mixture was then concentrated to about 0.3 mL, the residue was distributed between 50 mL ethyl acetate and 15 mL 0.1 N aqueous KHSO₄, the organic layer was then washed sequentially with 3×15 mL 0.1 M KHSO₄, 2×15 mL saturated NaHCO₃, 3×10 mL brine, and dried with anhydrous MgSO₄. The organic layer was then filtered, evaporated to dryness, and the residue was used in next step without further purification. The residue was dissolved in 5 mL anhydrous CH₂Cl₂, stirred, and cooled to −78° C. under Ar followed by addition of BCl₃ in CH₂Cl₂ (1.0 M, 3 mL, 3 mmol). The reaction was stirred at −78° C. for 1 hr followed by evaporation to dryness under high pressure, then co-evaporated to dryness with 2×5 mL anhydrous methanol, the residue was distributed between 10 mL water and 20 mL ethyl ether, and the aqueous layer was then isolated by HPLC. The target product D (R—CH₃) was collected, concentrated, and lyophilized to dryness.

H-(S)-Trp-boroSar. ¹H NMR (D₂O) δ 2.30 (s, 2H, NCH₂B), 2.63 (s, 3H, NCH₃), 3.38-3.55 (m, 2H, Ar—CH₂), 4.84 (dd, J=6.6, 8.2 Hz, 1H, H₂NCHCO), 7.20-7.64 (m, 5H, Ar—H); ¹¹B NMR (D₂O) δ −6.69; LCMS m/z: 258.1 (MH⁺−18), 298.1 (MNa⁺). HRMS: calcd for C₁₃H₁₇BN₃O₂, [M−H₂O+H]⁺, 258.1414, found 258.1416.

H-(S)-Pro-boroSar. ¹H NMR (D₂O) δ 2.04-2.57 (m, 4H, NHCH₂CH₂CH₂), 2.58 (s, 2H, NCH₂B), 3.16 (s, 3H, NCH₃), 3.40-3.51 (m, 2H, NHCH₂CH₂CH₂), 4.85 (br, 1H, H₂NCHCO); ¹¹B NMR (D₂O) δ −3.65; LCMS m/z: 169.2 (MH⁺−18), 209.2 (MNa⁺). HRMS: calcd for C₇H₁₄BN₂O₂, [M−H₂O+H]⁺, 169.1148, found 169.1155.

H-(S)-Glu-boroSar. ¹H NMR (D₂O) δ 2.21 (dt, J=6.4, 7.1 Hz, 2H, CH₂CO₂H), 2.50 (s, 2H, NCH₂B), 2.59 (t, J=7.1 Hz, 2H, CH₂CH₂CO₂H), 3.22 (s, 3H, NCH₃), 4.69 (t, J=6.4 Hz, 1H, H₂NCHCO); ¹¹B NMR (D₂O) δ −6.78; LCMS m/z: 201.1 (MH⁺−18). HRMS: calcd for C₇H₁₄BN₂O₄, [M−H₂O+H]⁺, 201.1047, found 201.1043.

H-Gly-boroSar. ¹H NMR (D₂O) δ 2.55 (s, 2H, NCH₂B), 3.10 (s, 3H, NCH₃), 4.18 (s, 2H, H₂NCH₂CO); ¹¹B NMR (D₂O) δ −4.37; LCMS m/z: 129.2 (MH⁺−18).

H-(S)-Val-boroSar. ¹H NMR (D₂O) δ 1.05 (d, J=6.9 Hz, 6H, CH(CH₃)₂), 2.32 (dq, J=6.9, 6.5 Hz, 1H, CH₃CHCH), 2.50 (d, J=14.8 Hz, 1H, NHCHB), 2.57 (d, J=14.8 Hz, 1H, NHCHB), 3.23 (s, 3H, NCH₃), 4.42 (d, J=6.5 Hz, 2H, H₂NCHCO); ¹¹B NMR (D₂O) δ −7.57; LCMS m/z: 171.1 (MH⁺−18). HRMS: calcd for C₇H₁₆BN₂O₂, [M−H₂O+H]⁺, 171.1305, found 171.1312.

H-(S)-Aad-boroSar. ¹H NMR (D₂O) δ 1.67-1.75 (m, 2H, CH₂CH₂CO₂H), 1.94-2.02 (m, 2H, CH₂CH₂CO₂H), 2.46 (t, J=7.1 Hz, 2H, CH₂CH₂CH₂CO₂H), 2.52 (d, J=14.8 Hz, 1H, NHCHB), 2.58 (d, J=14.8 Hz, 1H, NHCHB), 3.21 (s, 3H, NCH₃), 4.62 (t, J=6.4 Hz, 1H, H₂NCHCO); ¹¹B NMR (D₂O) δ −6.66; LCMS m/z: 215.1 (MH⁺−18).

H-(S)-Arg-boroSar. ¹H NMR (D₂O) δ 1.64-1.76 (m, 2H, NHCH₂CH₂CH₂), 1.96-2.04 (m, 2H, NHCH₂CH₂CH₂), 2.57 (s, 2H, NCH₂B), 3.22 (s, 3H, NCH₃), 3.25 (t, J=6.7 Hz, 2H, NHCH₂CH₂CH₂), 4.66 (t, J=6.5 Hz 1H, H₂NCHCO); ¹¹B NMR (D₂O) δ −5.87; LCMS m/z: 228.1 (MH⁺−18), 246.1 (MNa⁺). HRMS: calcd for C₈H₁₉BN₅O₂, [M−H₂O₊H]⁺, 228.1632, found 228.1626.

H-(S)-Ala(1-naph)-boroSar. ¹H NMR (D₂O) δ 2.08 (s, 3H, NCH₃), 2.16 (d, J=14.9 Hz, 1H, NHCHB), 2.25 (d, J=14.9 Hz, 1H, NHCHB), 3.60 (dd, J=10.9, 13.7 Hz, 1H, Ar—CH₂), 3.91 (dd, J=13.7, 5.4 Hz, 1H, Ar—CH₂), 4.91 (dd, J=5.4, 10.9 Hz, 1H, H₂NCHCO), 7.43-7.73 (m, 4H, Ar—H), 7.97-8.13 (m, 3H, Ar—H); ¹¹B NMR (D₂O) δ −6.51; LCMS m/z: 269.1 (MH⁺−18), 309.1 (MNa⁺). HRMS: calcd for C₁₅H₁₈BN₂O₂, [M−H₂O+H]⁺, 269.1461, found 269.1451.

H-(S)-Chg-boroSar. ¹H NMR (D₂O) δ 1.08-2.00 (m, 11H, cyclohexyl protons), 2.51 (d, J=14.9 Hz, 2H, NHCH₂B), 3.23 (s, 3H, NCH₃), 4.40 (d, J=6.8 Hz, 1H, H₂NCHCO); ¹¹B NMR (D₂O) δ −7.69; LCMS m/z: 211.1 (MH⁺−18), 251.1 (MNa⁺). HRMS: calcd for C₁₀H₂₀BN₂O₂, [M−H₂O+H]⁺, 211.1618, found 211.1628.

H-(S)-Ile-boroSar. ¹H NMR (D₂O) δ 0.94 (t, J=6.7 Hz, 3H, CH₂CH₃), 1.04 (d, J=7.0 Hz, 3H, CHCH₃), 1.21-1.60 (m, 2H, CH₂CH₃), 2.07-2.12 (m, 1H, CHCHNH₂), 2.54 (dd, J=14.9, 7.9 Hz, 2H, NHCHB), 3.24 (s, 3H, NCH₃), 4.47 (d, J=6.4 Hz, 1H, H₂NCHCO); ¹¹B NMR (D₂O) δ −7.39; LCMS m/z: 185.1 (MH⁺−18). HRMS: calcd for C₈H₁₈BN₂O₂, [M−H₂O+H]⁺, 185.1461, found 185.1458.

H-(S)-tert-Leu-boroSar. ¹H NMR (D₂O) δ 1.09 (s, 9H, 3×CH₃, t-butyl protons), 2.46 (d, J=14.9 Hz, 2H, NHCH₂B), 3.24 (s, 3H, NCH₃), 4.39 (s, 1H, H₂NCHCO); ¹¹B NMR (D₂O) δ −8.52; LCMS m/z: 185.2 (MH⁺−18), 225.1 (MNa⁺). HRMS: calcd for C₈H₁₈BN₂O₂, [M−H₂O+H]⁺, 185.1461, found 185.1466.

H-(S)-EthylGly-boroSar. ¹H NMR (D₂O) δ 1.03 (t, J=7.5 Hz, 3H, CH₃), 1.96-2.01 (m, 2H, CH₂CH₃), 2.53 (s, 2H, NCH₂B), 3.21 (s, 3H, NCH₃), 4.79 (t, J=6.6 Hz, 1H, H₂NCHCO); ¹¹B NMR (D₂O) δ −6.89; LCMS m/z: 157.2 (MW-18). HRMS: calcd for C₆H₁₄BN₂O₂, [M−H₂O+H]⁺, 157.1148, found 157.1153.

H-(S)-Phe-boroSar. ¹H NMR (D₂O) δ 2.42 (s, 2H, NCH₂B), 2.65 (s, 3H, NCH₃), 3.17-3.35 (m, 2H, Ar—CH₂), 4.77 (br, 1H, H₂NCHCO), 7.30-7.42 (m, 5H, Ar—H); ¹¹B NMR (D₂O) δ −6.71; LCMS m/z: 219.1 (MH⁺−18), 259.1 (MNa⁺). HRMS: calcd for C₁₁H₁₆BN₂O₂, [M−H₂O+H]⁺, 219.1305, found 219.1305.

H-(S)-Lys-boroSar. ¹H NMR (D₂O) δ 1.43-1.56 (m, 2H, H₂NCH₂CH₂CH₂), 1.66-1.77 (m, 2H, H₂NCH₂CH₂CH₂), 1.94-2.03 (m, 2H, H₂NCHCH₂), 2.50 (s, 2H, NCH₂B), 3.00 (t, J=7.1 Hz, 2H, H₂NCH₂CH₂CH₂) 3.22 (s, 3H, NCH₃), 4.63 (t, J=6.6 Hz, 1H, H₂NCHCO); ¹¹B NMR (D₂O) δ −6.93; LCMS m/z: 200.1 (MH⁺−18). HRMS: calcd for C₈H₁₉BN₃O₂, [M−H₂O+H]⁺, 200.1570, found 200.1565.

H-(S)-Met-boroSar. ¹H NMR (D₂O) δ 2.12 (s, 3H, SCH₃), 2.24 (dt, J=6.9, 7.0 Hz, 2H, CH₃SCH₂CH₂), 2.55 (s, 2H, NCH₂B), 2.66 (t, J=7.0 Hz, 2H, CH₃SCH₂CH₂), 3.24 (s, 3H, NCH₃), 4.76 (br, 1H, H₂NCHCO); ¹¹B NMR (D₂O) δ −5.94; LCMS m/z: 203.1 (MH⁺−18), 243.0 (MNa⁺). HRMS: calcd for C₇H₁₆BN₂O₂S, [M−H₂O+H]⁺, 203.1025, found 203.1028.

H-(S)-Tyr-boroSar. ¹H NMR (D₂O) δ 2.42 (s, 2H, NCH₂B), 2.69 (s, 3H, NCH₃), 3.09-3.29 (m, 2H, Ar—CH₂), 4.72 (dd, J=6.6, 9.4 Hz, 1H, H₂NCHCO), 6.90 (d, J=8.3 Hz, 2H, Ar—H), 7.17 (d, J=8.3 Hz, 2H, Ar—H); ¹¹B NMR (D₂O) δ −6.73; LCMS m/z: 235.2 (MH⁺−18). HRMS: calcd for C₁₁H₁₆BN₂O₃, [M−H₂O+H]⁺, 235.1254, found 235.1252.

H-(S)-Ala-boroSar. ¹H NMR (D₂O) δ 1.56 (d, J=6.4 Hz, 3H, CH₃CHNH₂), 2.53 (s, 2H, NCH₂B), 3.19 (s, 3H, NCH₃), 4.63 (q, J=6.4 Hz, 1H, H₂NCHCO); ¹¹B NMR (D₂O) δ −6.69; LCMS m/z: 143.1 (MH⁺−18). HRMS: calcd for C₅H₁₂BN₂O₂, [M−H₂O+H]⁺, 143.0992, found 143.0997.

H-(S)-n-PropylGly-boroSar. ¹H NMR (D₂O) δ 0.94 (t, J=7.3 Hz, 1H, CH₃CH₂), 1.35-1.46 (m, 2H, CH₃CH₂CH₂), 1.89 (dt, J=7.0, 8.8 Hz, 2H, CH₃CH₂CH₂), 2.53 (s, 1H, NCH₂B), 3.20 (s, 3H, NCH₃), 4.58 (t, J=6.7 Hz, 1H, H₂NCHCO); ¹¹B NMR (D₂O) δ −7.21; LCMS m/z: 171.1 (MH⁺−18), 211.1 (MNa⁺). HRMS: calcd for C₇H₁₆BN₂O₂, [M−H₂O+H]⁺, 171.1305, found 171.1309.

H-(S)-Thr-boroSar. ¹H NMR (D₂O) δ 1.32 (d, J=6.5 Hz, 3H, CH₃CHOH), 2.57 (d, J=5.2 Hz, 2H, NCH₂B), 3.26 (s, 3H, NCH₃), 4.26 (dt, J=5.8, 6.5 Hz, 1H, H₂NCHCHOH), 4.53 (d, J=5.8 Hz, 1H, H₂NCHCO). ¹¹B NMR (D₂O) δ −6.22; LCMS m/z: 173.1 (MH⁺−18), 213.1 (MNa⁺).

H-(S)-Leu-boroSar. ¹H NMR (D₂O) δ 0.99 (d, J=5.3 Hz, 6H, 2×CH₃), 1.72-1.86 (m, 3H, CHCH₂CHNH₂), 2.54 (s, 2H, NCH₂B), 3.21 (s, 3H, NCH₃), 4.58 (dd, J=5.3, 3.2 Hz, 1H, H₂NCHCO); ¹¹B NMR (D₂O) δ −6.54; LCMS m/z: 185.1 (MH⁺−18). HRMS: calcd for C₈H₁₈BN₂O₂, [M−H₂O+H]⁺, 185.1461, found 185.1472.

H-(S)-Gln-boroSar. ¹H NMR (D₂O) δ 2.11-2.20 (m, 2H, CH₂CONH₂), 2.41 (s, 2H, NCH₂B), 2.40-2.66 (m, 2H, CH₂CH₂CONH₂), 3.18 (s, 3H, NCH₃), 4.91 (dd, J=8.5, 4.7 Hz, 1H, H₂NCHCO); ¹¹B NMR (D₂O) δ −9.37.

H-(S)-Ser-boroSar. ¹H NMR (D₂O) δ 2.51 (s, KH, NCH₂B), 3.22 (s, 3H, NCH₃), 4.0 (m, 2H, HOCH2), 4.71 (dd, J=5.0, 5.6 Hz, 1H, H₂NCHCO); ¹¹B NMR (D₂O) δ −5.46; LCMS m/z: 159.0 (MH⁺−18), 199.0 (MNa⁺). HRMS: calcd for C₅H₁₂BN₂O₃, [M−H₂O+H]⁺, 159.0941, found 159.0748.

H-(S)-Asn-boroSar. ¹H NMR (D₂O) major isomer (˜6/7) δ 2.58 (s, 2H, NCH₂B), 2.93-3.00 (m, 2H, H₂NCOCH₂), 3.21 (s, 3H, NCH₃), 4.90 (dd, J=6.0, 7.4 Hz, 1H, H₂NCHCO); minor isomer (˜1/7): δ 5.00 (t, J=6.1 Hz); ¹¹B NMR (D₂O) δ −4.66, −18.0; LCMS m/z: 186.1 (MH⁺−18).

H-(S)-Asp-boroSar. ¹H NMR (D₂O) major isomer (˜2/3) δ 2.45 (d, J=14.5 Hz, 1H, NHCH₂B), 2.63 (d, J=14.5 Hz, 1H, NCH₂B), 2.79 (d, J=18.3 Hz, 1H, CH₂CO₂H), 2.94 (s, 3H, NCH₃), 3.05 (d, J=18.3 Hz, 1H, CH₂CO₂H), 4.28 (d, J=7.2 Hz, 1H, H₂NCHCO); minor isomer (˜1/3) δ 2.58 (s, 2H, NCH₂B), 3.00-3.20 (m, 2H, CH₂CO₂H), 3.45 (s, 3H, NCH₃), 4.91 (dd, J=5.5, 7.9 Hz, 1H, H₂NCHCO); ¹¹B NMR (D₂O) δ −4.73, −18.0; LCMS m/z: 187.0 (MH⁺−18).

H-(S)-His-boroSar. ¹H NMR (D₂O) major isomer (˜3/5) δ 2.34 (d, J=14.5 Hz, 2H, NCH₂B), 2.84 (d, J=8.5 Hz, 1H, CH₂CHCONH), 2.98 (d, J=14.5 Hz, 1H, NCH₂B), 2.86 (s, 3H, NCH₃), 3.32 (d, J=8.5 Hz, 1H, CH₂CHCONH), 4.40 (s, 1H, H₂NCHCO), 7.20 (s, 1H, HNCCHN), 8.34 (s, 1H, HNCHN); minor isomer (˜2/5): δ 2.60 (s, 1H, NCH₂B), 3.09 (s, 1H, CH₂CHCONH), 3.33 (s, 3H, NCH₃), 3.85 (s, 1H, CH₂CHCONH), 4.93 (t, 1H, H₂NCHCO), 7.51 (s, 1H, HNCCHN), 8.74 (s, 1H, HNCHN); ¹¹B NMR (D₂O) δ −20.6; LCMS m/z: 209.1 (MH⁺−18). HRMS: calcd for C₈H₁₄BN₄O₂, [M−H₂O+H]⁺, 209.1210, found 209.1203.

H-Aib-boroSar. ¹H NMR (D₂O) δ 1.76 (s, 6H, 2×CH₃), 2.57 (s, 2H, NCH₂B), 3.31 (s, 3H, NCH₃); ¹¹B NMR (D₂O) δ −9.15; LCMS m/z: 157.1 (MH⁺−18).

H-N-(Me)-Gly-boroSar. ¹H NMR (D₂O) δ 2.56 (s, 2H, NCH₂B), 2.81 (s, 3H, CH₃NCH₂CO), 3.08 (s, 3H, BCH₂NCH₃), 4.25 (s, 2H, CH₃NCH₂CO); ¹¹B NMR (D₂O) 6-2.94; LCMS m/z: 143.1 (MH⁺−18). HRMS: calcd for C₅H₁₂BN₂O₂, [M−H₂O+H]⁺, 143.0992, found 143.0998.

H-N-(Et)-Gly-boroSar. ¹H NMR (D₂O) δ 1.32 (t, J=7.3 Hz, 3H, CH₃CH₂), 2.57 (s, 2H, NCH₂B), 3.09 (s, 3H, NCH₃), 3.20 (q, J=7.3 Hz, 2H, CH₃CH₂N), 4.26 (s, 2H, NCH₂CO); ¹¹B NMR (D₂O) δ −3.37; LCMS m/z: 157.1 (MH⁺−18). HRMS: calcd for C₆H₁₄BN₂O₂, [M−H₂O+H]⁺, 157.1148, found 157.1150.

H-N-(tBu)-Gly-boroSar. ¹H NMR (D₂O) δ 1.40 (s, 9H, 3×CH₃), 2.56 (s, 2H, NCH₂B), 3.12 (s, 3H, NCH₃), 4.22 (s, 2H, NCH₂CO); ¹¹B NMR (D₂O) δ −3.81; LCMS m/z: 185.1 (MH⁺−18). HRMS: calcd for C₈H₁₈BN₂O₂, [M−H₂O+H]⁺, 185.1461, found 185.1462.

H-(S)-N-(Me)-Ala-boroSar. ¹H NMR (D₂O) δ 1.57 (d, J=7.0 Hz, 3H, CH₃NCHCH₃), 2.56 (s, 2H, NCH₂B), 2.74 (s, 3H, CH₃NCHCH₃), 3.19 (s, 3H, CH₃NCH₂B), 4.54 (q, 1H, J=7.0 Hz, H₂NCHCO); ¹¹B NMR (D₂O) δ −5.50; LCMS m/z: 157.1 (MH⁺−18). HRMS: calcd for C₆H₁₄BN₂O₂, [M−H₂O+H]⁺, 157.1148, found 157.1152.

General Procedure for the Synthesis of Dipeptide N-EthylboroGlycine (D, R═CH₂CH₃). Following the same procedure as described above the following compounds were obtained.

H-(S)-Trp-N(Et)boroGly. ¹H NMR (D₂O) δ 0.72 (t, J=7.2 Hz, 3H, NCH₂CH₃), 2.22-2.39 (m, 2H, NCH₂B), 2.82-3.14 (m, 2H, NCH₂CH₃), 3.37-3.54 (m, 2H, Ar—CH₂), 4.81-4.87 (m, 1H, H₂NCHCO), 7.01-7.63 (m, 5H, Ar—H); ¹¹B NMR (D₂O) δ −7.85; LCMS m/z: 272.2 (MH⁺−18), 312.1 (MNa⁺).

H-(S)-Pro-N(Et)boroGly. ¹H NMR (D₂O) δ 1.22 (t, J=7.2 Hz, 3H, NCH₂CH₃), 2.01-2.59 (m, 6H, NHCH₂CH₂CH₂, NCH₂B), 3.37-3.53 (m, 4H, NCH₂CH₃, NHCH₂CH₂CH₂), 4.85 (br, 1H, H₂NCHCO); LCMS m/z: 183.1 (MH⁺−18).

H-(S)-Glu-N(Et)boroGly. ¹H NMR (D₂O) δ 1.26 (t, J=7.2 Hz, 3H, NCH₂CH₃), 2.19-2.24 (m, 2H, CH₂CO₂H), 2.46 (s, 2H, NCH₂B), 2.58 (t, J=7.1 Hz, 2H, CH₂CH₂CO₂H), 3.50-3.64 (m, 2H, NCH₂CH₃), 4.70 (t, J=6.4 Hz, 1H, H₂NCHCO); ¹¹B NMR (D₂O) δ −6.44; LCMS m/z: 215.1 (MH⁺−18)

H-Gly-N(Et)boroGly. ¹H NMR (D₂O) δ 1.22 (t, J=7.2 Hz, 3H, NCH₂CH₃), 2.51 (s, 2H, NCH₂B), 3.43 (q, J=7.2 Hz, 2H, NCH₂CH₃), 4.22 (s, 2H, H₂NCH₂CO); ¹¹B NMR (D₂O) δ −6.23; LCMS m/z: 183.1 (MNa⁺), 143.1 (MH⁺−18).

H-(S)-Val-N(Et)boroGly. ¹H NMR (D₂O) δ 1.06 (d, J=6.8 Hz, 6H, CH(CH₃)₂), 1.26 (t, J=7.2 Hz, 3H, NCH₂CH₃), 2.27-2.34 (m, 1H, CH₃CHCH), 2.50 (s, 2H, NHCH₂B), 3.51-3.66 (m, 2H, NCH₂CH₃), 4.43 (d, J=6.4 Hz, 2H, H₂NCHCO); LCMS m/z: 185.1 (MH⁺−18).

H-(S)-Aad-N(Et)boroGly. ¹H NMR (D₂O) δ 1.26 (t, J=7.2 Hz, 3H, NCH₂CH₃), 1.67-1.75 (m, 2H, CH₂CH₂CO₂H), 1.94-2.02 (m, 2H, CH₂CH₂CO₂H), 2.47 (t, J=7.1 Hz, 2H, CH₂CH₂CH₂CO₂H), 2.50 (s, 2H, NHCH₂B), 3.55 (q, J=7.2 Hz, 2H, NCH₂CH₃), 4.64 (t, J=6.4 Hz, 1H, H₂NCHCO); LCMS m/z: 229.1 (MH⁺−18).

H-(S)-Arg-N(Et)boroGly. ¹H NMR (D₂O) δ 1.25 (t, J=7.2 Hz, 3H, NCH₂CH₃), 1.64-1.74 (m, 2H, NHCH₂CH₂CH₂), 1.95-2.03 (m, 2H, NHCH₂CH₂CH₂), 2.52 (s, 2H, NCH₂B), 3.24 (t, J=6.7 Hz, 2H, NHCH₂CH₂CH₂), 3.56 (q, J=7.2 Hz, 2H, NCH₂CH₃), 4.65 (t, J=6.5 Hz 1H, H₂NCHCO); LCMS m/z: 242.2 (MH⁺−18).

H-(S)-Ala(1-naph)-N(Et)boroGly. ¹H NMR (D₂O) δ 0.45 (t, J=7.2 Hz, 3H, NCH₂CH₃), 1.86 (d, J=14.8 Hz, 1H, NHCHB), 2.25-2.36 (m, 2H, NHCHB, NCH₂CH₃), 2.60-2.72 (m, 1H, NCH₂CH₃), 3.56-3.94 (m, 2H, Ar—CH₂), 4.95 (dd, J=5.2, 10.7 Hz, 1H, H₂NCHCO), 7.42-7.69 (m, 4H, Ar—H), 7.94-8.06 (m, 3H, Ar—H); ¹¹B NMR (D₂O) δ −7.59; LCMS m/z: 283.2 (MH⁺−18), 323.2 (MNa⁺).

H-(S)-Chg-N(Et)boroGly. ¹H NMR (D₂O) δ 1.11-1.64 (m, 14H, NCH₂CH₃, cyclohexyl protons), 2.49 (s, 2H, NHCH₂B), 3.50-3.66 (m, 2H, NCH₂CH₃), 4.41 (d, J=6.8 Hz, 1H, H₂NCHCO); LCMS m/z: 225.2 (MH⁺−18).

H-(S)-Ile-N(Et)boroGly. ¹H NMR (D₂O) δ 0.93 (t, J=7.4 Hz, 3H, NCH₂CH₃), 1.03 (d, J=6.9 Hz, 3H, CHCH₃), 1.28-1.59 (m, 5H, CH₂CH₃), 2.05-2.11 (m, 1H, CHCHNH₂), 2.50 (s, 2H, NHCH₂B), 3.51-3.65 (m, 2H, NCH₂CH₃), 4.46 (d, J=6.5 Hz, 1H, H₂NCHCO); ¹¹B NMR (D₂O) δ −8.21; LCMS m/z: 199.2 (MH⁺−18). H-(S)-tert-Leu-N(Et)boroGly. ¹H NMR (D₂O) δ 1.11 (s, 9H, 3×CH₃, t-butyl protons), 1.23-1.32 (m, 3H, NCH₂CH₃), 2.42-2.56 (m, 2H, NHCH₂B), 3.51-3.79 (m, 2H, NCH₂CH₃), 4.42 (s, 1H, H₂NCHCO); ¹¹B NMR (D₂O) δ −9.57; LCMS m/z: 199.1 (MH⁺−18).

H-(S)-EthylGly-N(Et)boroGly. ¹H NMR (D₂O) δ 1.01-1.03 (m, 3H, NCH₂CH₃), 1.14-1.26 (m, 3H, CHCH₂CH₃), 1.95-2.05 (m, 2H, CHCH₂CH₃), 2.50-2.60 (m, 2H, NCH₂B), 3.50-3.70 (m, 2H, NCH₂CH₃), 4.55-4.65 (m, 1H, H₂NCHCO); LCMS m/z: 171.1 (MH⁺−18).

H-(S)-Phe-N(Et)boroGly. ¹H NMR (D₂O) δ 0.88 (t, J=7.2 Hz, 3H, NCH₂CH₃), 2.30-2.46 (m, 2H, NCH₂B), 2.92-3.35 (m, 4H, NCH₂CH₃, Ar—CH₂), 4.72-4.83 (m, 1H, H₂NCHCO), 7.04-7.83 (m, 5H, Ar—H); ¹¹B NMR (D₂O) δ −7.70; LCMS m/z: 233.2 (MH⁺−18), 273.1 (MNa⁺).

H-(S)-Lys-N(Et)boroGly. ¹H NMR (D₂O) δ 1.25 (t, J=7.2 Hz, 3H, NCH₂CH₃), 1.44-1.53 (m, 2H, H₂NCH₂CH₂CH₂), 1.66-1.76 (m, 2H, H₂NCH₂CH₂CH₂), 1.94-2.03 (m, 2H, H₂NCHCH₂), 2.45-2.56 (m, 2H, NCH₂B), 2.99 (t, J=7.6 Hz, 2H, H₂NCH₂CH₂CH₂) 3.49-3.60 (m, 2H, NCH₂CH₃), 4.63 (t, J=6.5 Hz, 1H, H₂NCHCO); LCMS m/z: 214.1 (MH⁺−18).

H-(S)-Tyr-N(Et)boroGly. ¹H NMR (D₂O) δ 0.89 (t, J=7.2 Hz, 3H, NCH₂CH₃), 2.21-2.45 (m, 2H, NCH₂B), 2.89-3.28 (m, 4H, NCH₂CH₃, Ar—CH₂), 4.71-4.75 (m, 1H, H₂NCHCO), 6.89 (d, J=8.4 Hz, 2H, Ar—H), 7.17 (d, J=8.4 Hz, 2H, Ar—H); ¹¹B NMR (D₂O) δ −7.82; LCMS m/z: 249.2 (MH⁺−18).

H-(S)-Ala-N(Et)boroGly. ¹H NMR (D₂O) δ 1.24 (t, J=7.1 Hz, 3H, NCH₂CH₃), 1.55 (d, J=7.0 Hz, 3H, CH₃CHNH₂), 2.48 (s, 2H, NCH₂B), 3.52 (q, J=7.1 Hz, 2H, NCH₂CH₃), 4.62-4.77 (m, 1H, H₂NCHCO); LCMS m/z: 157.1 (MH⁺−18).

H-(S)-n-PropylGly-N(Et)boroGly. ¹H NMR (D₂O) δ 0.95 (t, J=7.3 Hz, 1H, CH₃CH₂), 1.38 (t, J=7.1 Hz, 3H, NCH₂CH₃), 1.41-1.48 (m, 2H, CH₃CH₂CH₂), 1.86-1.93 (m, 2H, CH₃CH₂CH₂), 2.49 (s, 1H, NCH₂B), 3.53 (q, J=7.1 Hz, 2H, NCH₂CH₃), 4.59 (t, J=6.6 Hz, 1H, H₂NCHCO); LCMS m/z: 185.1 (MH⁺−18).

H-(S)-Leu-N(Et)boroGly. ¹H NMR (D₂O) δ 0.97 (d, J=5.2 Hz, 6H, 2×CH₃), 1.27 (t, J=7.2 Hz, 3H, NCH₂CH₃), 1.69-1.73 (m, 2H, CHCH₂CHNH₂), 1.83-1.86 (m, 1H, CHCH₂CHNH₂), 2.45-2.51 (m, 2H, NCH₂B), 3.53 (q, J=7.2 Hz, 2H, NCH₂CH₃), 4.56-4.60 (m, 1H, H₂NCHCO); ¹¹B NMR (D₂O) δ −7.54; LCMS m/z: 199.2 (MH⁺−18), 239.1 (MNa⁺).

H-(S)-Gln-N(Et)boroGly. ¹H NMR (D₂O) δ 1.24 (t, J=7.2 Hz, 3H, NCH₂CH₃), 2.11-2.66 (m, 7H, NCH₂B, CH₂CH₂CONH₂), 3.52 (q, J=7.2 Hz, 3H, NCH₂CH₃), 4.93 (dd, J=8.8, 4.7 Hz, 1H, H₂NCHCO); ¹¹B NMR (D₂O) δ −9.37.

H-(S)-Ser-N(Et)boroGly. ¹H NMR (D₂O) δ 1.25 (t, J=7.2 Hz, 3H, NCH₂CH₃), 2.51 (s, 2H, NCH₂B), 3.56 (q, J=7.2 Hz, 2H, NCH₂CH₃), 3.96-3.99 (m, 2H, HOCH2), 4.70 (t, J=5.3 Hz, 1H, H₂NCHCO); ¹¹B NMR (D₂O) δ −6.53.

H-(S)-Asn-N(Et)boroGly. ¹H NMR (D₂O) δ 1.27 (t, J=7.2 Hz, 3H, NCH₂CH₃), 2.67 (s, 2H, NCH₂B), 3.28 (d, J=6.3 Hz, 2H, H₂NCOCH₂), 3.50-3.65 (m, 2H, NCH₂CH₃), 5.00 (t, J=6.3 Hz, 1H, H₂NCHCO); ¹¹B NMR (D₂O) δ −2.00.

H-(S)-Asp-N(Et)boroGly. ¹H NMR (D₂O) two isomers (˜1:1) δ 1.07 (t, J=7.2 Hz, 1.5H, NCH₂CH₃), 1.26 (t, J=7.2 Hz, 1.5H, NCH₂CH₃), 2.42-2.63 (m, 2H, NHCH₂B), 2.95-3.15 (m, 2H, CH₂CO₂H), 3.25-3.50 (m, 1H, NCH₂CH₃), 3.50-3.65 (m, 1H, NCH₂CH₃), 4.05-4.10 (m, 0.5H, H₂NCHCO), 4.85-4.95 (m, 0.5H, H₂NCHCO); LCMS m/z: 201.0 (MH⁺−18).

H-(S)-His-N(Et)boroGly. ¹H NMR (D₂O) two isomers (˜2:1) δ 0.88 (t, J=7.2 Hz, 2H, NCH₂CH₃), 1.12 (t, J=7.2 Hz, 1H, NCH₂CH₃), 2.40-2.85 (m, 2H, NHCH₂B), 3.16-3.48 (m, 4H, CH₂CHCONH, NCH₂CH₃), 4.38 (t, J=3.8 Hz, 0.65H, H₂NCHCO), 4.88 (t, J=3.8 Hz, 0.35H, H₂NCHCO), 7.20 (s, 0.65H, HNCCHN), 7.51 (s, 0.35H, HNCCHN), 8.39 (s, 0.65H, HNCHN), 8.74 (s, 0.35H, HNCHN); ¹¹B NMR (D₂O) δ −20.3; LCMS m/z: 223.1 (MH⁺−18).

H-Aib-N(Et)boroGly. ¹H NMR (D₂O) δ 1.29 (t, J=7.2 Hz, 3H, NCH₂CH₃), 1.77 (s, 6H, 2×CH₃), 2.53 (s, 2H, NCH₂B), 3.66 (q, J=7.2 Hz, 2H, NCH₂CH₃); ¹¹B NMR (D₂O) δ −8.84; LCMS m/z: 171.1 (MH⁺−18).

H-N-(Me)-Gly-N(Et)boroGly. ¹HNMR (D₂O) δ 1.21 (t, J=7.2 Hz, 3H, NCH₂CH₃), 2.51 (s, 2H, NCH₂B), 2.81 (s, 3H, CH₃NCH₂CO), 3.42 (q, J=7.2 Hz, 2H, NCH₂CH₃), 4.29 (s, 2H, CH₃NCH₂CO); ¹¹B NMR (D₂O) δ −5.10; LCMS m/z: 157.1 (MH⁺−18).

H-N-(Et)-Gly-N(Et)boroGly. ¹H NMR (D₂O) δ 1.21 (t, J=7.3 Hz, 3H, NCH₂CH₃), 1.31 (t, J=7.3 Hz, 3H, NHCH₂CH₃), 2.50 (s, 2H, NCH₂B), 3.19 (q, J=7.3 Hz, 2H, NHCH₂CH₃), 3.43 (q, J=7.2 Hz, 2H, NCH₂CH₃), 4.28 (s, 2H, NCH₂CO); LCMS m/z: 171.1 (MH⁺−18).

### Example 4

### DPP IV Inhibition Assay

Dipeptidyl peptidase IV was purified from human placenta. Assays were performed at room temperature (25° C.) in 100 mM HEPES, pH 8.0, 0.14 M NaCl with the chromogenic substrate Ala-Pro-p-nitroanalide (Bachem) using a Molecular Devices SPECTRAmax 340PC³⁸⁴ microtiter plate reader Inhibitor stocks (1 mg/mL) were prepared in 0.01 N HCl solution for pH 2.0 and in pH 8.0 HEPES buffer for pH 8.0. Both stocks were equilibrated at corresponding pH overnight at room temperature. The 1:10 dilution of inhibitor stocks was done with 0.01 N HCl for pH 2.0 and with pH 8.0 HEPES buffer for pH 8.0 immediately preceding the enzymatic reactions. Enzymatic reactions were carried out by incubating 250 μL of approximately 0.35 nM DPP IV with 30 μL of various inhibitor concentrations (ranging between 10⁻³ and 10⁻¹⁰ M) for 10 minutes before addition of 30 μL of 300 μM substrate. The absorbance at 410 nm was measured 30 minutes after addition of the substrate. Assays and their corresponding measurements were performed in triplicate. The negative control was prepared exactly the same as above, except that 30 μL of 0.01 N HCl was added instead of inhibitor solutions.

### Example 5

### Determination of IC₅₀ Values

After a 10 minute incubation with the enzyme before addition of the substrate. The IC₅₀ values were determined by fitting the data to a sigmoidal dose-response curve using non-linear regression in Prism 3.0 (Graph Pad).

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

All of the U.S. patents and U.S. patent application publications cited herein are hereby incorporated by reference.

1. A compound represented by: 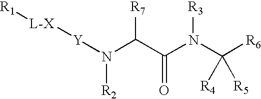
or a pharmaceutically acceptable salt thereof;
wherein
R₁ is selected from the group consisting of H, alkyl, alkoxy, alkenyl, alkynyl, amino, alkylamino, acylamino, cyano, sulfonylamino, acyloxy, aryl, cycloalkyl, heterocyclyl, heteroaryl, and polypeptide chains of 1 to 8 amino acid residues;
R₂ is selected from the group consisting of H, lower alkyl, and aralkyl;
R₃ is selected from the group consisting of
lower alkyl;
R₃₁R₃₂N(CH₂)ₘ—, wherein R₃₁ is a pyridinyl or pyrimidinyl moiety optionally mono- or independently disubstituted with (C₁₋₄)alkyl, (C₁₋₄)alkoxy, halogen, trifluoromethyl, cyano, or nitro; or phenyl optionally mono- or disubstituted with (C₁₋₄)alkyl, (C₁₋₄)alkoxy or halogen; and R₃₂ is selected from hydrogen and (C₁₋₈)alkyl; and m is 2 or 3;
(C₃₋₁₂)cycloalkyl optionally monosubstituted in the 1-position with (C₁₋₃)hydroxyalkyl;
R₃₃(CH₂)ₙ—, wherein either R₃₃ is selected from the group consisting of phenyl optionally mono- or independently di- or independently trisubstituted with (C₁₋₄)alkyl, (C₁₋₄)alkoxy, halogen, or phenylthio optionally monosubstituted in the phenyl ring with hydroxymethyl or (C₁₋₈)alkyl; [3.1.1]-bicyclic carbocyclic moiety optionally substituted with (C₁₋₈)alkyl; pyridinyl or naphthyl moiety optionally mono- or disubstituted with (C₁₋₄)alkyl, (C₁₋₄)alkoxy or halogen; cyclohexene; and adamantyl; and n is 1 to 3; or R₃₃ is phenoxy optionally mono- or disubstituted with (C₁₋₄)alkyl, (C₁₋₄)alkoxy or halogen; and n is 2 or 3;
(R₃₄)₂CH(CH₂)₂—, wherein R₃₄ independently is phenyl optionally mono- or disubstituted with (C₁₋₄)alkyl, (C₁₋₄)alkoxy or halogen;
(R₃₅)₂(CH₂)ₚ—, wherein R₃₅ is 2-oxopyrrolidinyl or (C₂₋₄)alkoxy and p is 2 to 4; and
R₃₆ is selected from indanyl; a pyrrolidinyl or piperidinyl moiety optionally substituted with benzyl; a [2.2.1]- or [3.1.1]bicyclic carbocyclic moiety optionally substituted with (C₁₋₈)alkyl; adamantyl; and (C₁₋₈)alkyl optionally substituted with hydroxy, hydroxymethyl, or phenyl optionally substituted with (C₁₋₄)alkyl, (C₁₋₄)alkoxy, or halogen;
R₄ is selected from the group consisting of H, halogen, and lower alkyl;
R₅ is selected from the group consisting of H, halogen, lower alkyl, and aralkyl;
R₆ is selected from the group consisting of —CN, —CH═NR₆₃, 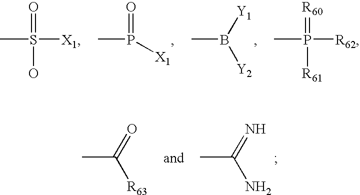
wherein
R₆₃ represents H, alkyl, alkenyl, alkynyl, —C(X₁)(X₂)(X₃), —(CH₂)ₘ—R₆₄, —(CH₂)ₙ—OH, —(CH₂)ₙ—O-alkyl, —(CH₂)ₙ—O-alkenyl, —(CH₂)ₙ—O-alkynyl, —(CH₂)ₙ—O—(CH₂)ₘ—R₆₄, —(CH₂)ₙ—SH, —(CH₂)ₙ—S-alkyl, —(CH₂)ₙ—S-alkenyl, —(CH₂)ₙ—S-alkynyl, —(CH₂)ₙ—S—(CH₂)ₘ—R₆₄, —C(O)C(O)NH₂, or —C(O)C(O)OR₆₅;
R₆₄ represents independently for each occurrence a substituted or unsubstituted aryl, aralkyl, cycloalkyl, cycloalkenyl, or heterocycle;
R₆₅ represents independently for each occurrence hydrogen, or a substituted or unsubstituted alkyl, alkenyl, aryl, aralkyl, cycloalkyl, cycloalkenyl, or heterocycle;
Y₁ and Y₂ independently represent OH or a group capable of being hydrolyzed to a hydroxyl group; or taken together with the boron to which they are bonded form a 5-membered 5 to 8-membered ring comprising said boron and two oxygen atoms bonded to said boron;
R₆₀ is O or S;
R₆₁ is N₃, SH, NH₂, NO₂ or —OR₇;
R₆₂ is selected from the group consisting of hydrogen, lower alkyl, amine, and —OR₆₅, or a pharmaceutically acceptable salt; or R₆₁ and R₆₂ taken together with the phosphorous atom to which they are attached complete a heterocyclic ring having from 5 to 8 atoms in the ring structure;
X₁ is halogen;
X₂ and X₃ is hydrogen or halogen; and
m is zero or an integer in the range of 1 to 8; and
n is an integer in the range of 1 to 8;
R₇ is selected from the group consisting of alkyl, alkoxy, alkenyl, alkynyl, aminoalkyl, aminoacyl, acyloxy, aryl, aralkyl, cycloalkyl, heterocyclyl, heteroaryl, and heteroaralkyl;
R₈ is selected from the group consisting of H, aryl, alkyl, aralkyl, cycloalkyl, heterocyclyl, heteroaryl, heteroaralkyl, and polypeptide chains of 1 to 8 amino acid residues;
L is absent or selected from the group consisting of alkyl, alkenyl, alkynyl, (CH₂)ₘ—O—(CH₂)ₘ—, —(CH₂)ₘNR₂(CH₂)ₘ—, and —(CH₂)ₘS(CH₂)ₘ—, wherein m is, independently for each occurrence, an integer from 0 to 10; and n is an integer from 1 to 6;
X is absent or selected from the group consisting of —N(R₈)—, —O—, and —S—; and
Y is absent or is selected from —C(═O)—, —C(═S)—, and —SO₂—.

2. The compound of claim 1, wherein R₆ is CN, CHO, or is C(X₁)(X₂)(X₃).

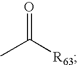

3. The compound of claim 1, wherein R₆ is 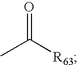
R₆₃ is C(X₁)(X₂)(X₃); X₁ is fluorine; and X₂ and X₃ are independently selected from the group consisting of H and fluorine.

4. The compound of claim 1, wherein R₆ is a group of formula —B(Y₁)(Y₂), wherein Y₁ and Y₂ independently represent OH or a group capable of being hydrolyzed to a hydroxyl group; or taken together with the boron to which they are bonded form a 5-membered to 8-membered ring comprising said boron and two oxygen atoms bonded to said boron.

5. The compound of claim 1, wherein R₃ is lower alkyl.

6. The compound of claim 5, wherein R₃ is selected from methyl, ethyl, and isopropyl.

7. The compound of claim 1, wherein the compound is a protease inhibitor.

8. The inhibitor of claim 7, wherein the protease inhibitor inhibits DPIV with a Kᵢ of 50 nm or less.

9. The compound of claim 1, wherein said compound is orally active.

10. A compound represented by the formula: 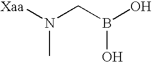
or a pharmaceutically acceptable salt thereof;
wherein Xaa is selected from the group consisting of Trp, Pro, Glu, Gly, Val, Aad, Arg, 1-Naphthyl-Ala, Chg (cyclohexylglycine), Ile, t-Leu, Ethly-Gly, Phe, Lys, Met, Tyr, Ala, n-Propyl-Gly, Thr, Leu, Gln, Ser, Asn, Asp, His, Methyl-Gly, Ethyl-Gly, t-Butyl-Gly, Methyl-Ala, Aib, N-Methyl-Gly, N-Ethyl-Gly, N-t-Butyl-Gly, and N-Methyl-Ala.

11. The compound of claim 10, wherein Xaa is selected from the group consisting of Pro, Gly, Ethyl-Gly, and N-Ethyl-Gly.

12. A compound represented by the formula: 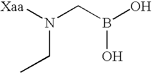
or a pharmaceutically acceptable salt thereof;
wherein Xaa is selected from the group consisting of Trp, Pro, Glu, Gly, Val, Aad, Arg, 1-Naphthyl-Ala, Chg (Cyclohexylglycine), Ile, t-Leu, Ethyl-Gly, Phe, Lys, Tyr, Ala, n-Propyl-Gly, Leu, Gln, Ser, Asn, Asp, His, Aib, N-Methyl-Gly, and N-Ethyl-Gly.

13. The compound of claim 12, wherein Xaa is selected from the group consisting of Pro, N-Methyl-Gly, and N-Ethyl-Gly.

14. A pharmaceutical composition, comprising a pharmaceutically acceptable carrier; and a compound of claim 1.

15-18. (canceled)

19. A method of inhibiting the proteolytic activity of a post-proline protease enzyme, comprising contacting the enzyme with a compound of claim 1.

20-23. (canceled)
